(19) United States
(12) Patent Application Publication
(10) Pub. No.: US 2026/0167679 A1
(43) Pub. Date: Jun. 18, 2026

(54) FUSION PROTEIN TARGETING MITOCHONDRIA, METHOD OF MAKING AND USE THEREOF

(71) Applicant: UNIVERSITY OF ROCHESTER, Rochester, NY (US)

(72) Inventors: Andrew WOJTOVICH, Rochester, NY (US); Brandon BERRY, Auburn, NY (US); Shahaf PELEG, Rostock (DE); Minsoo KIM, Pittsford, NY (US); Andrea AMITRANO, Rochester, NY (US)

(21) Appl. No.: 18/249,858

(22) PCT Filed: Nov. 12, 2021
(86) PCT No.: PCT/US21/72377

§ 371 Date: Apr. 20, 2023

Related U.S. Application Data
(60) Provisional application No. 63/115,826, filed on Nov. 19, 2020.

(51) Int. Cl. C07K 14/37; A61K 38/00; A61N 5/06; C07K 14/47

(52) U.S. Cl. CPC C07K 14/37; A61N 5/06; C07K 14/47; A61K 38/00; A61N 2005/0662; C07K 2319/03; C07K 2319/07; C07K 2319/60

(57) ABSTRACT

A fusion protein comprises (1) a first moiety that targets and orients the fusion protein to mitochondria inner membrane and (2) a second moiety that provides light-activated proton pump function when integrate into the mitochondria inner membrane. The fusion protein can be used for modulating hypoxia signaling in a subject, revitalizing cells, modulating T cell function, improving feed efficiency, prolonging lifespan and treating cancer.

This application is a continuation-in-part application that is a national stage claiming priority from International Application No. PCT/US21/72377, filed on Nov. 12, 2021, which claims priority from U.S. Provisional Application No. 63/115,826, filed on Nov. 19, 2020.

This invention was made with government support under NS092558, NS115906 and CA242843 awarded by the National Institute of Health. The government has certain rights in the invention.

### FIELD

This application relates to the field of optogenetics and mitochondrial activation for therapeutic purposes, such as controlling cell functions, revitalizing cells and the amelioration of age-associated damage through optical control of mitochondrial metabolism.

### BACKGROUND

Mitochondrion are a semi-autonomous double-membrane-bound organelle found in most eukaryotic organisms. Mitochondria are the powerhouse of the cell. Mitochondria generate an electrochemical proton gradient known as the protonmotive force (PMF). The PMF is like a battery, in that potential energy is stored for eventual release to do work. The PMF is created by the electron transport chain (ETC) in the mitochondrial inner membrane (IM) when electrons from metabolic substrates from food are passed along the chain and protons are pumped from the mitochondrial matrix to the intermembrane space (IMS) as oxygen is consumed.

Mitochondrial dysfunction is implicated in a wide range of disease. Optogenetics uses light-sensitive proteins to control biological functions. If targeted to mitochondria, optogenetic tools may allow rapid and precise manipulation of the PMF by controlled exposure to light. The ability to control mitochondrial function through use of light presents an intervention point to treat disease and other negative phenomena.

### SUMMARY

One aspect of the present application relates to a fusion protein (referring to as “mitochondria ON” or “mtON” hereinafter). The mtON fusion protein comprises a first moiety that targets the fusion protein to the mitochondrial inner membrane, and a second moiety that comprises a light-activated proton pump, wherein the first moiety orients the light-activated proton pump in the direction to pump protons from the mitochondrial matrix to the inner membrane space.

In some embodiments, the first moiety of the mtON fusion protein comprises an amino acid sequence selected from the group consisting of the mitochondria targeting sequence and transmembrane domains of one of human mitochondrial inner membrane protein (IMMT), rat IMMT, and mouse IMMT. In some embodiments, the first moiety comprises SEQ ID NO:6.

In some embodiments, the second moiety of the mtON fusion protein comprises an amino acid sequence selected from the group consisting of the protein sequence of Mac and variants, Arch and variants, bacteriorhodopsin (bR) and delta rhodopsin (dR). In some embodiments, the second moiety comprises SEQ ID NO:10.

In some embodiments, the first moiety is linked to the second moiety through a peptide linker. In some embodiments the peptide linker comprises the sequence of pro-ala-gly.

In some embodiments, the mtON fusion protein further comprises a third moiety that functions as a detection marker.

In some embodiments, the mtON fusion protein comprises an amino acid sequence that is at least 80% homologous to SEQ ID NO:6 and wherein the second moiety comprises an amino acid sequence that is at least 80% homologous to SEQ ID NO:10. In some embodiments, the mtON fusion protein comprises the amino acid sequence of SEQ ID NO:11.

Another aspect of the present application relates to a polynucleotide encoding the mtON fusion protein of the present application. In some embodiments, the polynucleotide comprises the nucleotide sequence of SEQ ID NO:12.

Another aspect of the present application relates to an expression cassette comprising a polynucleotide encoding the mtON fusion protein and a regulatory sequence operably linked to the polynucleotide.

Another aspect of the present application relates to an expression vector comprising a polynucleotide encoding the mtON fusion protein.

Another aspect of the present application relates to mitochondria containing the mtON fusion protein.

Another aspect of the present application relates to a cell containing mitochondria that contains the mtON fusion protein.

Another aspect of the present application relates to a pharmaceutical composition comprising an expression vector capable of expressing the mtON fusion protein and a pharmaceutically acceptable carrier.

Another aspect of the present application relates to a method of enhancing cell resistance to hypoxia in a subject. The method comprises the steps of (1) expressing the mtON fusion protein in target cells in the subject, and (2) exposing the target cells to light to activate the proton pump to increase the protonmotive force (PMF) across the mitochondrial inner membrane, wherein increased PMF in the mitochondria of the target cells enhances the target cells' resistance to hypoxia. In some embodiments, the mtON fusion protein is expressed in the target cells by infecting the target cells with a viral vector capable of expressing the mtON fusion protein in the target cells.

Another aspect of the present application relates to a method of ameliorating mitochondrial dysfunction in a subject. The method comprises the steps of (1) expressing the mtON fusion protein in target cells in the subject, and (2) exposing the target cells to light to activate the proton pump to increase the PMF across the mitochondrial inner membrane, wherein increased PMF in the mitochondria of the target cells reverses mitochondrial dysfunction in the target cells. In some embodiments, the mitochondrial dysfunction is age-related mitochondrial dysfunction. In some embodiments, the target cells comprise hair follicle cells or keratinocytes.

Another aspect of the present application relates to a method of treating cancer in a subject. The method comprise the steps of (1) expressing the mtON fusion protein in T cells in the subject, and (2) exposing mtON-expressing T cells to light to activate the proton pump to increase the PMF across the mitochondrial inner membrane, wherein increased PMF in the mitochondria of the mtON-expressing T cells enhances cancer killing activity of the T cells. In some embodiments, the T cells are chimeric antigen receptor (CAR)-T cells. In some embodiments the T cells are CD8⁺ T cells.

### BRIEF DESCRIPTION OF THE DRAWINGS

FIG. 1 is a drawing showing a fusion protein (mtON) of the present application. The fusion protein is a mitochondria-targeting, light-activated proton pump (mtON) that comprises a mitochondria-targeting sequence (MTS) and light-activated proton pump (LAPP). The MTS targets the mitochondria inner membrane and allows the LAPP to be incorporated into the mitochondrial inner membrane in an orientation that allows the proton pump to pump protons from the mitochondrial matrix to the intermembrane space.

FIG. 2 is a composite of drawings and pictures showing the expression of a mtON fusion protein of the present application. Panel A) Schematic depicting the targeting strategy to localize mtON to the mitochondrial inner membrane (IM). The electron transport chain (ETC) complexes generate the endogenous mitochondrial PMF by proton pumping, represented by the + and − across the IM. Mitochondrial ATP synthase utilizes the PMF to convert ADP to ATP. The N-terminal mitochondria target sequence from the IMMT1 protein and the C-terminal green fluorescence protein (GFP) marker are shown in hatched sections. In response to light, mtON pumps protons from the mitochondrial matrix to the intermembrane space (IMS). B) Confocal images demonstrate overlap of GFP tagged mtON with MitoTracker™ Red CMXRos stained C. elegans hypodermal mitochondria. Scale bar 10 μm. C) Immunoblot comparing the cytosolic supernatant and the mitochondria-enriched pellet of isolation fractions. GFP-tagged mtON migrates at the predicted molecular weight of 82 kDa accounting for the mitochondria-target sequence, the proton pump, and GFP. mtON is observed only in the mitochondrial fraction compared to marker proteins HSP60 (mitochondria) and actin (cytosol). All blots are from the same lanes on one membrane.

FIG. 3 is a composite of drawings showing that mtON activation increases the mitochondrial protonmotive force (PMF). Panel A) Representative TMRE fluorescence traces in arbitrary units (a.u.) before and after Δψm dissipation with FCCP. Dashed lines indicate where FCCP was added. +/−ATR traces were performed in the absence of succinate. mtON activation was continuous throughout the traces. Light green trace is from mitochondria with ATR, gray traces are without. Panel B) Quantification of change in fluorescence (ΔF) normalized to the maximum change in fluorescence given by succinate respiration (ΔFmax) for each mitochondrial preparation. Data are from the maximum light dose. Two-way ANOVA with Sidak's test for multiple comparisons, *p=0.0469, +ATR succinate vs. +ATR+light p=0.9978, n=6 mitochondrial isolations. Bars are means±SEM. Panel C) Representative BCECF-AM 490/440 nm ratio trace. Ratio of 545 nm fluorescence intensity at either 440 or 490 nm excitation. Dashed lines are where light or FCCP treatment occurred. Light green trace is from mitochondria with ATR, gray traces are without. Panel D) Quantification of change in BCECF-AM fluorescence ratio normalized to maximum change given by succinate matrix alkalization. Two-way ANOVA with Sidak's test for multiple comparisons, −ATR succinate vs. −ATR, +light*p=0.0212, +ATR succinate vs. +ATR light p=0.999, +ATR succinate vs. +ATR, −light p=0.0237, +ATR, +light vs, +ATR, −light p=0.0474, n=3 mitochondrial isolations. Some individual points overlap. Bars are means+SEM. Panel E) ATP levels normalized to total ATP synthesis given by succinate respiration. Succinate data shown for comparison after normalization. Two-way ANOVA with Sidak's test for multiple comparisons, *p=0.0011, +ATR succinate vs+ATR light p=0.5680, n=3, 7, 3, 7, for each bar from left to right. Some individual points overlap. Bars are means+SEM. Panel F) 02 required to consume 50 nmoles of ADP after mtON activation. One-way ANOVA*p=0.013 (−ATR, −light vs. +ATR, +light p=0.010. −ATR, +light vs. +ATR, +light p=0.013.)−ATR, −light n=5, rest n=6 mitochondrial preparations. Bars are means+SEM.

FIG. 4 is a composite of drawings showing that mtON reverses mitochondrial dysfunction. Panel A) Day 1 adult animals expressing mtON were exposed to 50 μM rotenone (ETC complex I inhibitor) for 5 hours and survival was scored. Illumination was continuous throughout toxin exposure. ATR alone was protective (−ATR, −light vs. +ATR, −light p=0.016.) The effect of mtON activation was greater than the ATR alone effect *p=0.01. (−ATR, −light vs +ATR, +light p=0.0002. −ATR, +light vs. +ATR, +light p=0.0009. n=3 plates each condition with at least 15 animals per plate). Bars are means±SEM. Panel B) mtON-expressing animals were exposed to 50 μM antimycin A (complex III inhibitor) and survival was scored 18 hours later. * p=0.02 (−ATR, −light vs+ATR, +light p=0.03, −ATR, +light vs. +ATR, +light p=0.01. n=5 plates each condition with at least 15 animals per plate). Bars are means±SEM. Panel C) mtON-expressing animals were exposed to 0.25 M azide (complex IV inhibitor) for 1 hour and scored for survival 1 hour after recovering. * p=0.0002. (−ATR, −light vs+ATR, +light p<0.0001, −ATR, +light vs. +ATR, +light p<0.0001. n=3 plates each condition with at least 15 animals per plate). Statistics are one-way ANOVA with Tukey's post hoc test. Bars are means±SEM. Panel D) mtON-expressing animals were exposed to 31 μM oligomycin A (ATP synthase inhibitor) and survival was scored 18 hours later. No significant differences were found by one-way ANOVA (n=3 plates each condition with at least 15 animals per plate). Bars are means±SEM. Panel E) mtON-expressing animals in a complex I mutant background (gas-1) were scored for locomotion speed in the presence of food by counting body bends per minute. mtON activation rescued the decreased locomotion of the gas-1 mutant background, one-way ANOVA with Tukey's post hoc test, −ATR, +light vs. +ATR, +light *p=0.0145, +ATR, +light vs. wildtype (WT) *p=0.0008 (−ATR, −light vs+ATR, +light p=0.0272, +ATR, −light vs. +ATR, +light p=0.0332, all conditions vs WT, p<0.001, n=30, 30, 30, 41, 41 animals each bar from left to right). Bars are means±standard deviation.

FIG. 5 is a composite of drawings showing that mtON affects whole-animal energy sensing. Panel A) AMPK is one way that an organism senses energy status. Top: Immunoblot assessing the effect of mtON activation on AMPK phosphorylation status. Top bands (˜62 kDa) are phosphorylated AMPK signal, and bottom bands (˜43 kDa) are actin signal. Image is from the same membrane cut to separately probe for phosphorylated AMPK (pAMPK) and actin. Bottom: Densitometry analysis showing decreased pAMPK to actin ratio, as there is no known antibody directed against total AMPK in C. elegans. Phosphorylation increases in the absence of food, but low phosphorylation is preserved when mtON is activated, two-way ANOVA with Sidak's test for multiple comparisons, *p=0.0203, n=3, 3, 3, 4, 4 biological replicates each bar from left to right. Bars are means±standard deviation. Panel B) Locomotion was assessed by counting body bends per minute. Wild type animals were compared to aak-2(ok524) mutant animals. 2-sample, 2-tailed unpaired t-test *p<0.0001, wild type n=35, aak-2 n=39 animals across at least 3 days. Bars are means±standard deviation. Panel C) Locomotion in response to mtON activation. Illumination was continuous throughout body bends measurement (see methods). For AAK-2 activation, animals were exposed to 1 mM AICAR (5-aminoimidazole-4-carboxamide ribonucleotide, AMPK activator) for 4 hours before body bends measurement. One-way ANOVA with Tukey's post hoc test, −ATR, +light vs+ATR, +light *p<0.0001, +ATR, +light vs. AICAR *p<0.0001, +ATR, +light vs AICAR+light *p<0.0001. n=36, 39, 37, 46, 36, 36 animals each bar from left to right. Bars are means±standard deviation.

FIG. 6 is a composite of drawings showing that mtON inhibits hypoxia-adaptation. Panel A) Schematic of HR experiments. Top shows control hypoxia and reoxygenation. Below is the hypoxic preconditioning (PC) protocol, survival for these two timelines shown in panel B. Third from the top shows FCCP treatment protocol, calculated protection shown in panel C. Fourth and fifth from the top show light treatment protocol+ and − PC, Calculated protection data from these protocols shown in panel D. Bottom shows light treatment during hypoxia, calculated protection data from these protocols shown in panel E. Panel B) Survival after HR in day 1 adult animals is shown with hypoxic preconditioning (PC, represented by diagonal stripes) 2-sample 2-tailed unpaired t-test, ctrl vs. PC*p=0.0035, n=4, each averaged from 3 technical replicates with at least 15 animals per replicate. Bars are means±SEM. Panel C) Protection (%) is percent survival minus percent survival of control condition. PC data calculated from panel B. FCCP final concentrations 0.001, 0.01, 0.1, 1 nM. One-way ANOVA with Tukey's post hoc test, PC vs. 0.001 nM FCCP, p=0.0012, 0.001 nM FCCP vs 0.01 FCCP, p=0.0142, n=4, 3, 3, 3, 3 independent experiments each bar from left to right, each averaged from 3 technical replicates with at least 15 animals per replicate. Bars are means±SEM. Panel D) Illumination was continuous throughout PC alone; control illumination was for the same duration under normoxic conditions (see methods & panel A). Two-way ANOVA comparing+ATR vs-ATR in each group. * p=0.016, n=11, 11, 4, 4, 6, 6 independent experiments each bar from left to right, each averaged from 3 technical replicates with at least 15 animals per replicate. Bars are means±SEM. Panel E) light during hypoxia. 2 sample 2-tailed unpaired t-test, *p=0.0276, n=7 independent experiments, each averaged from 3 technical replicates with at least 15 animals per replicate. Bars are means±SEM.

FIG. 7 is a composite of drawings showing that animals lose mitochondrial membrane potential with age. Panel A) Representative images of animals stained with mitochondrial (mito) membrane potential indicator (TMRE) and mitochondrial mass indicator (MitoTracker™ Green FM). Images were acquired at day 4 and day 10 of adulthood. All scale bars are 250 μm. Panel B) Quantification of panel A. Membrane potential is significantly decreased with age. TMRE fluorescence is used to quantify mitochondrial membrane potential (a.u. is arbitrary fluorescence units). Decreased TMRE fluorescence indicates decreased mitochondrial membrane potential. Increased MitoTracker™ Green FM fluorescence is membrane potential-independent, and thus reflects total mitochondrial mass (a.u. is arbitrary fluorescence units). The ratio of TMRE and MitoTracker™ Green FM signals (rightmost plot) indicates that mitochondrial membrane potential is decreased with age. One-way ANOVA with Tukey's post hoc test for multiple comparisons, *p<0.05. Data are means+standard error of the mean.

FIG. 8 is a composite of drawings showing that mtON restores lost membrane potential and increases lifespan. Panel A) mtON significantly increases aged mitochondrial membrane potential. The ratio of TMRE to MitoTracker™ Green FM fluorescence shows a significant increase in mitochondrial membrane potential after mtON activation for 4 days of adulthood. One-way ANOVA with Tukey's post hoc test for multiple comparisons, *p<0.05. Data are means±standard error of the mean. Panel B) mtON activation throughout adult life extends lifespan (n=?). Animals with mtON activity lived significantly longer than control populations. Log rank Mantel Cox test for survival comparison, p=0.018.

FIG. 9 shows characterization of mtOn (aka OptoMito-On). (A) Illustration of photoactivatable oxidative phosphorylation by mtOn construct. (B) Images of HeLa cells and CD8+ T cells expressing mtOn and stained with MitoTracker Red CMXRos. (C) Immunoblot comparing control HEK293T cell lysate and HEK293T mtOn cell lysate. The full-length mtOn construct is 82 kDa and probed for with an anti-GFP antibody. β-actin is used as loading control. Both images are from the same lanes on one membrane. (D) Representative TMRE fluorescence trace of isolated mitochondria from mtOn expressing HEK293T cells before and after addition of FCCP. Dashed lines indicate where FCCP was added. (E) Quantification of change in TMRE fluorescence. Data shown as mean±SEM and analyzed by One-Way ANOVA with a Bonferroni post-test (n=4).

FIG. 10 shows activation of mtOn increases CD8+ T cell migration. The velocity (A), displacement (B), track length (C), meandering index (D), and percent migrating cells (E) of Mock (or GFP) and mtOn expressing Day 4 CD8+ T cells migrating on ICAM-1+CXCL12. The percentage of migrating cells was calculated as the number of cells migrating 5-20 μm/min divided by the total number of cells in the field of view during the 20-minute movie. All movies received 500 nm illumination and were analyzed with Volocity software. All data shown as mean±SEM and analyzed by a two-tailed unpaired t-test with Welch's correction (A-D: includes four independent experiments, Mock: 271 cells, mtOn: 312 cells; E: n=4 movies on the same day). ns, not significant.

FIG. 11 shows mtON increased aged PMF in C. elegans. Panel A) Representative images of day 4 adult animals exposed to 100 nM TMRE for 24 hours. Pharynx region of interest (ROI) is outlined in dotted white lines. All animals were expressing mtON through single-copy CRISPR/Cas9 insertion to the genome. Panel B) Quantification of TMRE fluorescence in mtON-expressing day 4 adult animals exposed to 100 nM TMRE for 24 hours. Whole-body fluorescence as well as pharynx ROIs were quantified. Control represents animals exposed to light but without ATR supplementation. Statistics are student's two tailed unpaired t tests. * p<0.001.

FIG. 12 shows mtON optogenetic activation extended C. elegans lifespan. Panel A) Lifespan of mtON expressing worms under control conditions, no treatment versus ATR supplementation (cofactor). Panel B) Lifespan of mtON expressing worms under light-control conditions and activated mtON conditions, *p<0.05. Statistics are Logrank test for survival. Lifespan plots are presented as days from egg. mtON was activated throughout life starting at day 1 of adulthood. All animals were expressing mtON through single-copy CRISPR/Cas9 insertion to the genome.

FIG. 13 shows mtON is expressed in Drosophila melanogaster and colocalizes with a mitochondrial marker. On the left panel, we show that mtON-GFP is detected in the heads of mtON drosophila and that the GFP signal (eGFP) co-localizes with mitochondria (MitoTracker DeepRed). Control drosophila (w-1118) show only mitochondria staining but no GFP signal. Hoechst stains nuclei. On the right panel, as confirmed by PCR and sequencing, the full length mtON-GFP product is present and has the correct nucleotide sequence.

FIG. 14 shows mitochondria-ON increased Δψm in vivo. a, The mitochondrial inner membrane (IM) contains the electron transport chain (ETC), which pumps protons to generate mitochondrial membrane potential (Δψm). mtON is an engineered light-activated proton pump and, in response to light and ATR supplementation, pumps protons across the IM to generate Δψm. b, Adult worms stained with the Δψm indicator, TMRE. Scale bar, 250 μm. c, Quantification of relative TMRE fluorescence. One-way analysis of variance (ANOVA) with Tukey's multiple comparisons test, day 1 versus day 4, P=0.0001, day 1 versus day 10, P=0.0002, n=44, 18 and 30. Data are median±quartiles (dotted lines). d, Pharynx TMRE fluorescence normalized to mitochondrial mass in day 4 worms. One-way ANOVA with Tukey's test, all significant differences, P=0.0001, n=33, 34, 26 and 35 animals for each bar from left to right. Data are median±quartiles. e, Basal oxygen consumption of day 1 (left) and day 4 (right) animals, no ATR n=5 populations and ATR n=6 populations. Data are mean+s.e.m. Dots are individual populations. f, Maximal oxygen consumption (induced by FCCP) of the same populations (no ATR n=5 populations and ATR n=6 populations) in h. Data are mean±s.e.m. Dots are individual populations.

FIG. 15 shows mtON extended lifespan and healthspan. Light treatment began at day 1 of adulthood for all experiments, a, Survival curves of mtON-expressing animals (extrachromosomal array). Only mtON activation (+ATR+light) significantly extended lifespan, log-rank (Mantel-Cox) test, *P=0.019. Detailed statistical information for all lifespans is presented in FIG. 17. b, Survival curves of mtON-expressing animals (CRISPR insertion). mtON activation significantly extended lifespan compared to the light control by log-rank (Mantel-Cox) test, *P=0.0001, gray and light-green curves. c, Increased lifespan by mtON activation was sensitive to FCCP, log-rank (Mantel-Cox) test, P=0.0001. d, mtON activation did not affect locomotion on solid media. One-way ANOVA with Tukey's test; n=30 animals for day 4 conditions and 40 animals for day 10. Data are median±quartiles (dotted lines). e, mtON activation improved mobility in liquid with age. One-way ANOVA with Tukey's test, day 10 no ATR light versus day 10 ATR light; P=0.002, all other significant comparisons, P=0.0001; n=32, 32, 40 and 40 animals for each violin from left to right. Data are median±quartiles. f, Model showing effects of mtON activation in vivo. The dotted arrow represents the molecular mechanisms to be investigated that link Δψm to aged physiology.

FIG. 16 shows mtON has no effect at low light exposure and increases lifespan of animals with constitutively active AMPK signaling. A) survival curve with the same conditions as FIG. 15B, but with light intensity (0.005 mW/cm2) below the threshold for mtON activation (0.01 mW/cm2). With low light mtON (+ATR+light) no longer extends lifespan, log-rank (Mantel-Cox) test. B) Survival curves of mtON-expressing animals versus mtON-expressing animals in a long-lived genetic background containing a constitutively active AMPK mutation. Light treatment began at day 1 of adulthood. mtON activation in the CA-AMPK strain extended lifespan further compared to mtON alone (p=0.0001) and compared to constitutively active AMPK alone (p=0.0022), log-rank (Mantel-Cox) test. Detailed statistical information is presented in FIG. 17.

FIG. 17 shows mtON reproducibly extended lifespan. Strain APW32 expresses mtON as an extrachromosomal array (mtON ext. chr. array), and strain APW273 expresses mtON in single copy through CRISPR/Cas9 genome integration (mtON CRISPR). See methods section for genotype details. Light intensity was varied to maintain temperature as best as possible, and proximal temperature readings directly adjacent to assay plates are reported to show local temperature variability from different incubators and from different light sources. All incubators were set to 20° C. Bolded experiments correspond to the data displayed in FIG. 15 and in FIG. 16.

FIG. 18 shows confocal imaging of mtON in Drosophila muscle cells. Left panel: fluorescence of mtON::GFP showing a mitochondrial expression pattern. Middle panel: nuclear localized RFP. Right panel: merged image showing localization of mtON-GFP (green) outside the nucleus (red).

FIG. 19 shows (A) RT PCR of Drosophila heads showing induction of mtON transcript in crossed UAS-mtON×arm-GAL4 Drosophila compared with UAS-mtON. (B) In dark conditions ATR have similar lifespan compared to vehicle (ethanol, blue). (C) White light increases the lifespan of mtON+ATR (red) Drosophila compared to vehicle (ethanol, blue) Drosophila.

FIG. 20 shows activation of mtOn increases ATP production. (A) HEK293T cells expressing mtOn or GFP were illuminated with 590 nm light for 2 hours, followed by a luciferase-based ATP assay. (B) Same set-up as in (A), except HEK293T cells were treated with 10 mM 2-DG for 2 hours prior to illumination. (C) Activated CD8+ T cells were sorted based on GFP expression. GFP negative cells were used as the mock control. CD8+ T cells received 590 nm light for 30 minutes, followed by a luciferase-based ATP assay. All data shown as mean±SEM and analyzed by an unpaired t-test (n=4-7). This figure shows that the activation of mtON in HEK293T of CD8+ T cells can increase ATP production.

FIG. 21 shows mitochondrial membrane potential and mass measurements. A) Confocal images of animals stained separately with TMRE or MitoTracker Green, demonstrating no channel bleed-through. Scale bar is 10 μm. Experiment was performed 3 times with similar results. B) Confocal images of animals co-stained with TMRE and MitoTracker Green demonstrating mitochondrial colocalization. Scale bar is 10 μm. Experiment was performed 3 times with similar results. C) Relative pharynx TMRE fluorescence in mtON (ex. array) animals stained at day 4 of adulthood under the indicated conditions. One-way ANOVA with Tukey's test, *p=0.0001, n=33, 34, 26, 35 animals for each bar from left to right. D) Relative pharynx MitoTracker fluorescence in animals stained at day 4 of adulthood. One-way ANOVA, no significant differences detected between groups. n=33, 34, 26, 35 animals for each bar from left to right. Data from panels C and D were used for normalized mitochondrial membrane potential presented in FIG. 14, panel D.

FIG. 22 shows mtON activation did not affect mitochondrial mass or mitochondrial unfolded protein response by quantitative proteomics. A) The levels of key mitochondrial proteins were not significantly altered by 4 days of mtON activity, two-tailed unpaired t test, −ATR n=5, +ATR n=4 biological replicates. Genes encoding proteins are as follows. Complex I: nuo-1, nuo-5. complex II: sdha-1, mev-1, sdhb-1. Complex III: isp-1. Complex IV: cox-5a. ATP synthase: R04F11.2, atp-5. Translocase of the outer membrane (TOM70): tomm-70. VDAC: vdac-1. Uniprot C. elegans proteome database protein codes are presented above each plot. B) Proteins that mediate the mitochondrial unfolded protein response are not affected by mtON activation, two-tailed unpaired t test, −ATR n=5, +ATR n=4 biological replicates. Genes encoding proteins are hsp-6 and hsp-60.

While the present disclosure will now be described in detail, and it is done so in connection with the illustrative embodiments, it is not limited by the particular embodiments illustrated in the figures and the appended claims.

### DETAILED DESCRIPTION

Herein incorporated by reference is the sequence listing filed with the USPTO named as 1134-110 NATL-CIP_ST25.txt which was created on Nov. 6, 2023, and the size of the TXT file is 39,402 in bytes.

Reference will be made in detail to certain aspects and exemplary embodiments of the application, illustrating examples in the accompanying structures and figures. The aspects of the application will be described in conjunction with the exemplary embodiments, including methods, materials and examples, such description is non-limiting and the scope of the application is intended to encompass all equivalents, alternatives, and modifications, either generally known, or incorporated here. Unless otherwise defined, all technical and scientific terms used herein have the same meaning as commonly understood by one of ordinary skill in the art to which this application belongs. One of skill in the art will recognize many techniques and materials similar or equivalent to those described here, which could be used in the practice of the aspects and embodiments of the present application. The described aspects and embodiments of the application are not limited to the methods and materials described.

As used in this specification and the appended claims, the singular forms “a,” “an” and “the” include plural referents unless the content clearly dictates otherwise.

### Definitions and Terminology

As used herein, the following terms shall have the following meanings:

The term “polynucleotide” refers to deoxyribonucleotides or ribonucleotides and polymers thereof in either single- or double-stranded form. Unless specifically limited, the terms encompass nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences and as well as the sequence explicitly indicated. Specifically, degenerate codon substitutions can be achieved by generating sequences in which the third position of one or more selected (or all) codons is substituted with mixed-base and/or deoxyinosine residues. The term “polynucleotide” or “polynucleotide sequence” can also be used interchangeably with gene, open reading frame (ORF), cDNA, and mRNA encoded by a gene.

The terms “polypeptide”, “protein”, and “peptide”, which are used interchangeably herein, refer to a polymer of the 20 protein amino acids, or amino acid analogs, regardless of its size or function. Although “protein” is often used in reference to relatively large polypeptides, and “peptide” is often used in reference to small polypeptides, usage of these terms in the art overlaps and varies. The term “polypeptide” as used herein refers to peptides, polypeptides, and proteins, unless otherwise noted. The terms “protein”, “polypeptide” and “peptide” are used interchangeably herein when referring to a gene product. Thus, exemplary polypeptides include gene products, naturally occurring proteins, homologs, orthologs, paralogs, fragments and other equivalents, variants, and analogs of the foregoing.

The term “variant” refers to protein or polypeptide that is different from the reference protein or polypeptide by one or more amino acids, e.g., one or more amino acid substitutions, but substantially maintains the biological function of the reference protein or polypeptide. The term “variant” further includes conservatively substituted variants. The term “conservatively substituted variant” refers to a peptide comprising an amino acid residue sequence that differs from a reference peptide by one or more conservative amino acid substitution, and maintains some or all of the activity of the reference peptide as described herein. A “conservative amino acid substitution” is a substitution of an amino acid residue with a functionally similar residue. Examples of conservative substitutions include the substitution of one non-polar (hydrophobic) residue such as isoleucine, valine, leucine or methionine for another; the substitution of one charged or polar (hydrophilic) residue for another, such as between arginine and lysine, between glutamine and asparagine, between threonine and serine; the substitution of one basic residue such as lysine or arginine for another; or the substitution of one acidic residue, such as aspartic acid or glutamic acid for another; or the substitution of one aromatic residue, such as phenylalanine, tyrosine, or tryptophan for another. The phrase “conservatively substituted variant” also includes peptides wherein a residue is replaced with a chemically derivatized residue, provided that the resulting peptide maintains some or all of the activity of the reference peptide as described herein. In some embodiments, the functional variant of a peptide shares a sequence identity of 70%, 75%, 80%, 85%, 90%, 91%, 92%, 93%, 94%, 95%, 96%, 97%, 98% or 99% with the reference peptide. For example, a functional variant of a protein may share a sequence identity of 70%, 75%, 80%, 85%, 90%, 91%, 92%, 93%, 94%, 95%, 96%, 97%, 98% and 99% with the reference version of the protein; and a functional variant of a fusion protein may shares a sequence identity of 70%, 75%, 80%, 85%, 90%, 91%, 92%, 93%, 94%, 95%, 96%, 97%, 98% and 99% with the reference fusion protein.

A variant of a polypeptide may be a fragment of the original polypeptide. The term “fragment”, when used in reference to a reference polypeptide, refers to a polypeptide in which amino acid residues are deleted as compared to the reference polypeptide itself, but where the remaining amino acid sequence is usually identical to the corresponding positions in the reference polypeptide. Such deletions can occur at the amino-terminus or carboxy-terminus of the reference polypeptide, or alternatively both. Fragments typically are at least 3, 5, 6, 8 or 10 amino acids long, at least 14 amino acids long, at least 20, 30, 40 or 50 amino acids long, at least 75 amino acids long, or at least 100, 150, 200, or more amino acids long.

The term “homologous amino acid sequence” used in this specification, unless otherwise stated herein, refers to an amino acid sequence derived from the substitution of one or more amino acids in the amino acid sequence of a polypeptide. Furthermore, the term “homologous polypeptide” used in this specification, unless otherwise stated herein, refers to a polypeptide homologue derived from the substitution of one or more amino acids in the amino acid sequence of a polypeptide.

The term “sequence identity,” as used herein, means that two peptide sequences are identical (i.e., on an amino acid-by-amino acid basis) over the window of comparison. The term “percentage of sequence identity” is calculated by comparing two optimally aligned sequences over the window of comparison, determining the number of positions at which the identical amino acid residue occurs in both sequences to yield the number of matched positions, dividing the number of matched positions by the total number of positions in the window of comparison (i.e., the window size), and multiplying the result by 100 to yield the percentage of sequence identity. The reference sequence may be a subset of a larger sequence, for example, as a segment of the full-length sequences of the compositions claimed in the present invention.

The term “proton pump” means an integral membrane protein that is capable of moving protons across the membrane of a cell, mitochondrion, or other subcellular compartment. For example, bacteriorhodopsins are light-activated electrogenic proton pumps that are 7-transmembrane helix proteins (7-TM), utilize all-trans retinal as their chromophore in their native state, and bear structural similarity to the H. salinarum bacteriorhodopsin. Commonly characterized bacteriorhodopsins are the H. salinarum bacteriorhodopsin, the S. ruber xanthorhodopsin, and uncultured gamma-protobacterium BAC31A8. Other examples are microbial rhodopsins, such as the Halorubrum sodomense gene for archaerhodopsin-3 (herein abbreviated “Arch”) and Halorubrum strain TP009 gene for archaerhodopsin-TP009 (herein abbreviated “ArchT”), and eukaryotic proton pumps, such as leptosphaeria maculans (herein abbreviated “Mac”), P. triticirepentis, and S. sclerotorium rhodopsins.

The term “expression cassette,” as used herein, refers to a DNA or RNA construct that contains one or more transcriptional regulatory elements operably linked to a nucleotide sequence coding the fusion protein of the present application. An expression cassette may additionally contain one or more elements positively affecting mRNA stability and/or an internal ribosome entry site (IRES) between adjacent protein coding regions to facilitate expression two or more proteins from a common mRNA.

A nucleic acid sequence is “operably linked” to another nucleic acid sequence when the former is placed into a functional relationship with the latter. For example, a DNA for a presequence or signal peptide is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous and, in the case of a signal peptide, contiguous and in reading phase. However, enhancers do not have to be contiguous. Linking is accomplished by ligation at convenient restriction sites. If such sites do not exist, synthetic oligonucleotide adaptors or linkers may be used in accordance with conventional practice.

The term “regulatory elements” refers to DNA/RNA sequences necessary for the expression of an operably linked coding sequence in one or more host organisms. The term “regulatory elements” is intended to include promoters, enhancers and other expression control elements (e.g., polyadenylation signals). Regulatory elements include those which direct constitutive expression of a nucleotide sequence in many types of host cells or those which direct expression of the nucleotide sequence only in certain host cells (e.g., tissue-specific regulatory elements). Expression cassettes generally contain sequences for transcriptional termination, and may additionally contain one or more elements positively affecting mRNA stability.

As used herein, the term “promoter” is to be taken in its broadest context and includes transcriptional regulatory elements (TREs) from genomic genes or chimeric TREs therefrom, including the TATA box or initiator element for accurate transcription initiation, with or without additional TREs (i.e., upstream activating sequences, transcription factor binding sites, enhancers, and silencers) which regulate activation or repression of genes operably linked thereto in response to developmental and/or external stimuli, and trans-acting regulatory proteins or nucleic acids. A promoter may contain a genomic fragment or it may contain a chimera of one or more TREs combined together.

The term “expression vectors,” as used herein, refers to recombinant expression vectors comprising nucleic acid molecules which encode the fusion proteins disclosed herein. Particularly useful vectors are contemplated to be those vectors comprising the expression cassette of the present application or those vectors in which the coding portion of the DNA segment is positioned under the control of a regulatory element. The expression vectors of the present application is capable of expressing the fusion protein of the present application in a cell transfected or infected by the expression vector. Expression vectors include non-viral vectors and viral vectors.

The term “non-viral vector,” as used herein, refers to an autonomously replicating, extrachromosomal circular DNA molecules, distinct from the normal genome. For example, a plasmid is a non-viral vector.

The terms “viral vector” and “recombinant virus” are used interchangeably herein to refer to any of the obligate intracellular parasites having no protein-synthesizing or energy-generating mechanism. The viral genome may be RNA or DNA contained with a coated structure of protein of a lipid membrane. The viruses useful in the practice of the present invention include recombinantly modified enveloped or non-enveloped DNA and RNA viruses, preferably selected from baculoviridiae, parvoviridiae, picornoviridiae, herpesviridiae, poxviridae, or adenoviridiae. The viral genomes may be modified by recombinant DNA techniques to include expression of exogenous transgenes and may be engineered to be replication deficient, conditionally replicating or replication competent. Chimeric viral vectors which exploit advantageous elements of each of the parent vector properties may also be useful in the practice of the present application. Minimal vector systems in which the viral backbone contains only the sequences need for packaging of the viral vector and may optionally include a transgene expression cassette may also be produced according to the practice of the present application. Although it is generally favored to employ a virus from the species to be treated, in some instances it may be advantageous to use vectors derived from different species which possess favorable pathogenic features. A viral vector may be derived from an adeno-associated virus (AAV), adenovirus, herpesvirus, vaccinia virus, poliovirus, poxvirus, a retrovirus (including a lentivirus, such as HIV-1 and HIV-2), Sindbis and other RNA viruses, alphavirus, astrovirus, coronavirus, orthomyxovirus, papovavirus, paramyxovirus, parvovirus, picornavirus, togaviruses and the like.

The term “retrovirus” refers to double-stranded RNA enveloped viruses that are primarily characterized by the ability to “reverse transcribe” their genome from RNA to DNA. The virions are 100-120 nm in diameter and contain a dimeric genome of the same plus RNA strand complexed with the nucleocapsid protein. The genome is encapsulated in a proteic capsid that also contains the enzymatic proteins required for viral infection, namely reverse transcriptase, integrase and protease. Matrix proteins form the outer layer of the capsid core that surrounds the viral nuclear particle and interacts with the envelope, a lipid bilayer derived from the host cell membrane. Immobilized in this bilayer is a viral envelope glycoprotein that is responsible for recognizing specific receptors on the host cell and initiating the infectious process. Envelope proteins are formed by two subunits, a transmembrane (TM) that anchors the protein within the lipid membrane and a surface (SU) that binds to cell receptors.

Based on the genomic structure, retroviruses are classified into simple retroviruses such as MLV and murine leukemia virus; or complex retroviruses such as HIV and EIAV. Retroviruses encode four genes, gag (group-specific antigen), pro (protease), pol (polymerase) and env (envelope). The gag sequence encodes three major structural proteins: matrix protein, nucleocapsid protein, and capsid protein. The pro sequence encodes a protease responsible for cleaving Gag and Gag-Pol during particle assembly, budding and maturation. The pol sequence encodes the enzymes reverse transcriptase and integrase, the former catalyzing the reverse transcription of the viral genome from RNA to DNA during the infection process and the latter the role of incorporating proviral DNA into the host cell genome. Carry. The env sequence encodes both the SU and TM subunits of the envelope glycoprotein. In addition, the retroviral genome contains two LTRs (long terminal repeats) that contain the elements necessary to facilitate gene expression, reverse transcription and integration into the host cell chromosome; viral RNA into newly formed virions. A sequence designated as the packaging signal (Ψ) required for specific packaging; as well as a non-coding cis such as a polypurine tract (PPT) that functions as a site to initiate plus-strand DNA synthesis during reverse transcription. The acting sequence is presented. In addition to gag, pro, pol and env, complex retroviruses such as lentiviruses regulate viral gene expression, assembly of infectious particles and modulate vif, vpr, vpu, nef, which modulates viral replication in infected cells. It has accessory genes including tat and rev.

During the process of infection, retroviruses first attach to specific cell surface receptors. Upon entry into a susceptible host cell, the retroviral RNA genome is copied into DNA by the virally encoded reverse transcriptase carried within the parental virus. This DNA is transported to the host cell nucleus and then integrated into the host genome. At this stage it is typically called a provirus. Proviruses are stable in the host chromosome during cell division and are transcribed like other cellular proteins. Proviruses encode the proteins and packaging machinery required to make more virus and can leave the cell by a process known as “budding”.

The term “lentivirus” or “lentiviral vector” as used herein, refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

The term “adeno-associated virus (AAV)” or “recombinant AAV (rAAV),” as used herein, refers to a group of replication-defective, nonenveloped viruses, that depend on the presence of a second virus, such as adenovirus or herpes virus or suitable helper functions, for replication in cells. AAV is not known to cause disease and induces a very mild immune response. AAV can infect both dividing and non-dividing cells and may incorporate its genome into that of the host cell. More than 30 naturally occurring serotypes of AAV are available. Many natural variants in the AAV capsid exist, allowing identification and use of AAV vectors with properties specifically suited for the cell targets of delivery. AAV vectors are relatively nontoxic, provide efficient gene transfer, and can be easily optimized for specific purposes. AAV viruses may be engineered using conventional molecular biology techniques to optimize the generation of recombinant AAV particles for cell specific delivery of the fusion proteins, for minimizing immunogenicity, enhancing stability, delivery to the nucleus, etc.

The term “Car-T” refers to T cells modified to express a chimeric antigen receptor (CAR). T cells that have been genetically modified to express a CAR are used in treatments for cancers where the CAR redirects the modified T cell to recognize a tumor antigen. In some instances, it is beneficial to effectively control and regulate CAR T cells such that they kill tumor cells while not affecting normal bystander cells. The nucleic acid encoding CAR can be introduced into cells such as T cells using the retroviral vector or lentiviral vector. In this way, large numbers of cancer-specific T cells can be generated for adoptive cell transplantation methods. When CAR binds to the target antigen, an activating signal is transmitted to the T cells in which it is expressed. Thus, CAR dictates T cell specificity and cytotoxicity for tumor cells expressing the target antigen.

The term “mitochondria ON,” “mtON,” “mtON construct,” or “mtON protein” as used herein, refers to a fusion protein that comprises a first moiety that targets the fusion protein to the mitochondrial inner membrane and a second moiety that comprises a light-activated proton pump, wherein the first moiety orients the second moiety in a direction that allows the proton pump to pump protons from the mitochondrial matrix to the inner membrane space (mtON direction).

The term “mtON polynucleotide,” as used herein, refers to a polynucleotide comprising a sequence that encodes a mtON protein.

The term “mtON expression cassette,” as used herein, refers to an expression cassette comprising a mtON polynucleotide.

The term “mtON expression vector,” as used herein, refers to an expression vector capable of expressing a mtON protein inside a cell.

The term “mtON mitochondria,” as used herein, refers to a mitochondrion comprising one or more functional mtON protein on its inner membrane.

The term “mtON cell,” as used herein, refers to a cell comprising one or more mtON mitochondria.

The terms “treat,” “treating” or “treatment” as used herein, refers to a method of alleviating or abrogating a disorder and/or its attendant symptoms. The terms “prevent”, “preventing” or “prevention,” as used herein, refer to a method of barring a subject from acquiring a disorder and/or its attendant symptoms. In certain embodiments, the terms “prevent,” “preventing” or “prevention” refer to a method of reducing the risk of acquiring a disorder and/or its attendant symptoms.

The term “inhibits” is a relative term, an agent inhibits a response or condition if the response or condition is quantitatively diminished following administration of the agent, or if it is diminished following administration of the agent, as compared to a reference agent. Similarly, the term “prevents” does not necessarily mean that an agent completely eliminates the response or condition, so long as at least one characteristic of the response or condition is eliminated. Thus, a composition that reduces or prevents an infection or a response, such as a pathological response, can, but does not necessarily completely eliminate such an infection or response, so long as the infection or response is measurably diminished, for example, by at least about 50%, such as by at least about 70%, or about 80%, or even by about 90% of (that is to 10% or less than) the infection or response in the absence of the agent, or in comparison to a reference agent.

A “therapeutically effective amount,” as used herein, refers to an amount effective, at dosages and for periods of time necessary, to achieve the desired therapeutic result. A therapeutically effective amount of an expression vector may vary depending on the condition to be treated, the severity and course of the condition, the mode of administration, whether the agent is administered for preventive or therapeutic purposes, the bioavailability of the particular agent(s), the ability of the fusion protein or vector to elicit a desired response in the individual, previous therapy, the age, weight and sex of the patient, the patient's clinical history and response to the antibody, the type of the fusion protein or expression vector used, discretion of the attending physician, etc. A therapeutically effective amount is also one in which any toxic or detrimental effects of the expression vector is outweighed by the therapeutically beneficial effects. A “prophylactically effective amount” refers to an amount effective, at dosages and for periods of time necessary, to achieve the desired prophylactic result.

As used herein, the term “pharmaceutically acceptable carrier” includes any and all solvents, dispersion media, coatings, antibacterial and antifungal agents, isotonic and absorption delaying agents, and the like that are physiologically compatible. Pharmaceutical compositions may comprise suitable solid or gel phase carriers or excipients. Exemplary carriers or excipients include but are not limited to, calcium carbonate, calcium phosphate, various sugars, starches, cellulose derivatives, gelatin, and polymers such as polyethylene glycols. Exemplary pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the therapeutic agents.

The term “tumor” as used herein refers to a neoplasm or a solid lesion formed by an abnormal growth of cells. A tumor can be benign, pre-malignant or malignant.

The term “cancer” is defined as a malignant neoplasm or malignant tumor and is a class of diseases in which a group of cells display uncontrolled growth, invasion that intrudes upon and destroys adjacent tissues, and sometimes metastasis, or spreading to other locations in the body via lymph or blood. These three malignant properties of cancers differentiate them from benign tumors, which do not invade or metastasize. Exemplary cancers include: carcinoma, melanoma, sarcoma, lymphoma, leukemia, germ cell tumor, and blastoma.

As used herein, the term “inflammatory disorder” includes diseases or disorders which are caused, at least in part, or exacerbated, by inflammation, which is generally characterized by increased blood flow, edema, activation of immune cells (e.g., proliferation, cytokine production, or enhanced phagocytosis), heat, redness, swelling, pain and/or loss of function in the affected tissue or organ. The cause of inflammation can be due to physical damage, chemical substances, micro-organisms, tissue necrosis, cell proliferative disorders, or other agents.

As used herein, the term “subject” includes both human and animal subjects. Thus, veterinary therapeutic uses are provided in accordance with the presently disclosed subject matter.

Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economic importance to humans. Thus, also provided is the treatment of livestock, including, but not limited to, fish, domesticated swine, ruminants, ungulates, horses (including race horses), poultry, and the like.

The term “mammal” refers to any animal classified as a mammal, including humans, non-human primates, domestic and farm animals, and zoo, sports, or pet animals, such as dogs, horses, cats, cows, etc. Preferably, the mammal is human.

mtON Fusion Protein and mtON Expression Vectors
The mtON Fusion Protein

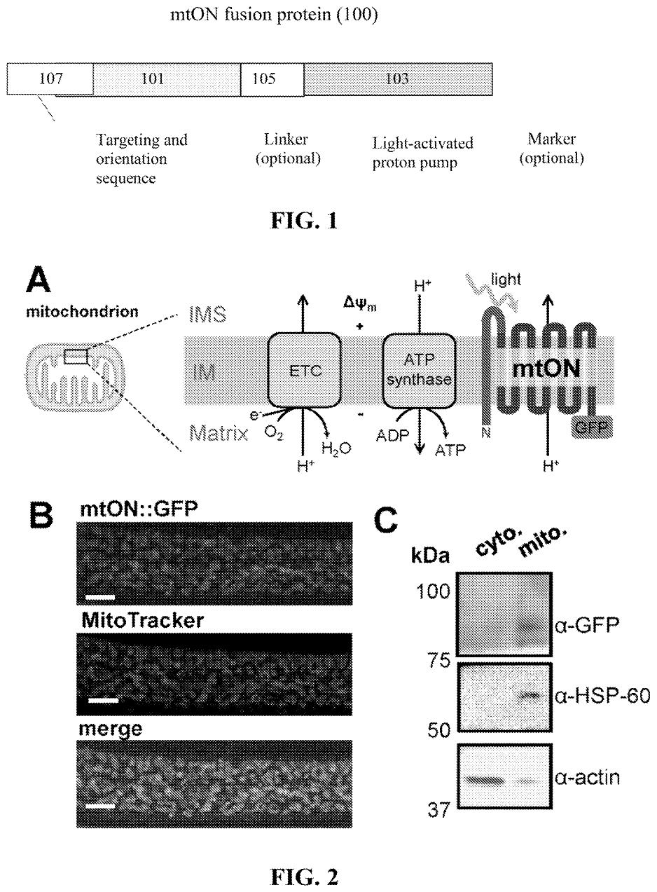

One aspect of the present application relates to a fusion protein. As shown in FIG. 1, the fusion protein 100 (mtON) comprises a first moiety 101 that targets the fusion protein to the mitochondrial inner membrane and a second moiety 103 that comprises a light-activated proton pump, wherein the first moiety orients the proton pump in the direction to pump protons from the mitochondrial matrix to the inner membrane space (mtON direction). In some embodiments, the first moiety and the second moiety are linked directly to each other. In some embodiments, the first moiety and the second moiety are linked to each other through a peptide linker 105. In some embodiments, the fusion protein 100 further comprises a marker 107 for easy localization of the fusion protein within a cell or a mitochondrion. In some embodiments, the fusion protein is expressed within mitochondria to reduce the likelihood of immune responses to the fusion protein.

The first moiety of mtON targets the fusion protein to the mitochondrial membrane and orient the fusion protein such that the proton pump pumps protons from the mitochondrial matrix to the inner membrane space. In some embodiments, the first moiety comprises a canonical mitochondrial targeting sequence and a generic transmembrane domain that orient the fusion protein such that the proton pump pumps protons from the mitochondrial matrix to the inner membrane space.

The mitochondrial targeting sequence can be any sequence capable of targeting the fusion protein to mitochondria membrane. Examples of mitochondrial targeting sequences include, but are not limited to, the mitochondrial targeting sequences of inner membrane mitochondrial proteins (IMMT), aspartate amino transferases, cytochrome c oxidase subunits (e.g. COX8A), citrate synthase, aconitases, ATP synthase subunits (e.g. ATP5A), TOM70, NADH ubiquinone oxoreductases and mitochondrial ATP-ase inhibitors.

The transmembrane domain can be any transmembrane domain that is capable of orienting the fusion protein in the mitochondria membrane such that the proton pump pumps protons from the mitochondrial matrix to the inner membrane space. Any mitochondrial protein, or fragment thereof, with its N-terminus in the matrix that spans the inner membrane of mitochondria in an odd number (e.g., 1, 3, 5, etc) may be sufficient to target and orient mtON.

In some embodiments, the first moiety comprises an amino acid sequence from human IMMT (SEQ ID NO:1) and variants thereof, mouse IMMT (SEQ ID NO:4) and variants thereof, or rat IMMT (SEQ ID NO:7) and variants thereof that is capable of targeting and orienting the mtON construct in the mitochondria membrane.

In some embodiments, the first moiety comprises the first 65 amino acids, first 66 amino acids, . . . up to the first 187 amino acids of the human IMMT or variants thereof. In some embodiments, the first moiety comprises the first 65 amino acids, first 66 amino acids, . . . up to the first 187 amino acids of the mouse IMMT or variants thereof. In some embodiments, the first moiety comprises the first 65 amino acids, first 66 amino acids, . . . up to the first 187 amino acids of the rat IMMT or variants thereof.

In some embodiments, the first moiety comprises the first 65 amino acids of human IMMT (SEQ ID NO:2) or variants thereof. In some embodiments, the first 187 amino acids of human IMMT (SEQ ID NO:3) or variants thereof.

In some embodiments, the first moiety comprises the first 65 amino acids of mouse IMMT (SEQ ID NO:5) or variants thereof, the first moiety comprises the first 187 amino acids of mouse IMMT (SEQ ID NO:6) or variants thereof.

In some embodiments, the first moiety comprises the first 66 amino acids of rat IMMT (SEQ ID NO:8) or variants thereof. In some embodiments, the first moiety comprises the first 187 amino acids of mouse IMMT (SEQ ID NO:9) or variants thereof.

In some embodiments, the first moiety comprises a sequence that is 80%, 85%, 90%, 95%, 96%, 97%, 98%, 99% or 100% identical to the first 187 amino acids of human IMMT (SEQ ID NO:3).

In some embodiments, the first moiety comprises a sequence that is 80%, 85%, 90%, 95%, 96%, 97%, 98%, 99% or 100% identical to the first 187 amino acids of mouse IMMT (SEQ ID NO:6).

In some embodiments, the first moiety comprises a sequence that is 80%, 85%, 90%, 95%, 96%, 97%, 98%, 99% or 100% identical to the first 187 amino acids of rat IMMT (SEQ ID NO:9).

The second moiety of mtON may be any canonical light-activated proton pump. Examples of light-activated proton pump include, but are not limited to, Mac (Leptosphaeria maculans rhodopsin) and variants, such as eMac3.0; Arch (Halorubrum sodomense archaerhodopsin-3) and variants, such as ArchT, eArch3.0m and eArchT3.0; bacteriorhodopsin (bR) and the related delta rhodopsin (dR).

In some embodiments, the second moiety comprises an amino acid sequence that is 80%, 85%, 90%, 95%, 96%, 97%, 98%, 99% or 100% identical to from Mac (SEQ ID NO:10) and variants thereof.

In some embodiments, the second moiety comprises the amino acid sequence of SEQ ID NO:10.

In some embodiments, the first moiety is joined to the second moiety directly. In other embodiments, the first moiety is linked to the second moiety by a peptide linker. In some embodiments, the linker comprises hydrophilic residues. In some embodiments, the linker is the remainder resulting from the restriction cloning used to generate the fusion. In some embodiments, the linker is Pro-Ala-Gly.

In some embodiments, the fusion protein of the present application comprises the amino acid sequence of SEQ ID NO:11. In other embodiments, the fusion protein of the present application further comprises a fluorescent protein marker, such as GFP.

Polynucleotide Encoding the mtON Fusion Protein

Another aspect of the present application relates to a polynucleotide encoding the fusion protein of the present application. In some embodiments, the polynucleotide encodes a fusion protein (mtON) that comprises a first moiety that targets the fusion protein to the mitochondrial inner membrane and a second moiety that comprises a light-activated proton pump, wherein the first moiety orients the proton pump in the direction to pump protons from the mitochondrial matrix to the inner membrane space.

In certain embodiments, the polynucleotide encodes a fusion protein which is mammalian codon optimized. In some embodiments, the polynucleotide of the present application further comprises a coding sequence for an amino terminal signal peptide, which is removed from the mature protein. Since the signal peptide sequences can affect the levels of expression, the polynucleotides may encode any one of a variety of different N-terminal signal peptide sequences. It will be appreciated by those skilled in the art that the design of the polynucleotide of the present application can depend on such factors as the choice of the host cell to be transformed, the level of expression of protein desired, and the like.

In some embodiments, the polynucleotide comprises a sequence encoding the fusion protein of SEQ ID NO:11. In some embodiments, the polynucleotide comprises the sequence of SEQ ID NO:12.

mtON Expression Cassette

Another aspect of the application relates to an expression cassette that comprises one or more regulatory sequences operably linked to the coding sequence of the fusion protein of the present application. The fusion protein (mtON) comprises a first moiety that targets the fusion protein to the mitochondrial inner membrane and a second moiety that comprises a light-activated proton pump, wherein the first moiety orients the proton pump in the direction to pump protons from the mitochondrial matrix to the inner membrane space.

In some embodiments, the one or more regulatory sequences include a promoter and a ′3 UTR sequence. Preferred promoters are those capable of directing high-level expression in a target cell of interest. The promoters may include constitutive promoters (e.g., HCMV, SV40, elongation factor-1α (EF-1α)) or those exhibiting preferential expression in a particular cell type of interest. In some embodiments, a ubiquitous promoter such as a CMV promoter or a CMV-chicken beta-actin hybrid (CAG) promoter to control the expression of the fusion protein of the present application. In other embodiments, a tissue specific promoter, such as skin specific promotor, neuron specific promotor, muscle specific promoter and liver specific promoter, is used to control the expression of the fusion protein in a specific tissue. Tissue specific promoters are well known in the art.

In some embodiments, it is contemplated that certain advantages will be gained by positioning the coding sequence under the control of a recombinant, or heterologous, promoter. As used herein, a recombinant or heterologous promoter is intended to refer to a promoter that is not normally associated with a protein's gene in its natural environment. Such promoters may include promoters isolated from plant, insect, bacterial, viral, eukaryotic, fish, avian or mammalian cells. Naturally, it will be important to employ a promoter that effectively directs the expression of the DNA segment in the cell type chosen for expression. The use of promoter and cell type combinations for protein expression is generally known to those of skill in the art of molecular biology.

In some embodiments, the one or more regulatory sequences further comprise an enhancer. Enhancers generally refer to DNA sequences that function away from the transcription start site and can be either 5′ or 3′ to the transcription unit. Furthermore, enhancers can be within an intron as well as within the coding sequence. They are usually between 10 and 300 bp in length, and they function in cis. Enhancers function to increase and/or regulate transcription from nearby promoters. Preferred enhancers are those directing high-level expression in the antibody producing cell.

In some embodiments, cell or tissue-specific transcriptional regulatory elements (TREs) can be incorporated into expression cassette to restrict expression to desired cell types. An expression vector may be designed to facilitate expression of the fusion proteins herein in one or more cell types.

In some embodiments, the expression cassette of the present application comprises a nucleotide sequence encoding the fusion protein of SEQ ID NO:11. In some embodiments, the expression cassette of the present application comprises the nucleotide sequence of SEQ ID NO:12.

mtON Expression Vectors

Another aspect of the present application relates to an expression vector comprising the expression cassette of the present application. The expression cassette comprises (1) a polynucleotide encoding a fusion protein comprising a first moiety that targets the fusion protein to the mitochondrial inner membrane and a second moiety that comprises a light-activated proton pump, wherein the first moiety orients the proton pump in the direction to pump protons from the mitochondrial matrix to the inner membrane space; and (2) a regulatory sequence operably linked to the polynucleotide.

### Non-Viral Vectors

In some embodiments, the expression vector is a non-viral expression vector. In some embodiments, the non-viral expression vector is a plasmid capable of expressing the fusion protein of the present application in an in vitro and/or in vivo setting.

In some embodiments, non-viral expression vectors of the present application are introduced into cells or tissues by encapsulating the expression vectors in liposomes, microparticles, microcapsules, virus-like particles, or erythrocyte ghosts. Such compositions can be further linked by chemical conjugation to, for example, microbial translocation domains and/or targeting domains to facilitate targeted delivery and/or entry of nucleic acids into the nucleus of desired cells to promote gene expression. In addition, plasmid vectors may be incubated with synthetic gene transfer molecules such as polymeric DNA-binding cations like polylysine, protamine, and albumin, and linked to cell targeting ligands such as asialoorosomucoid, insulin, galactose, lactose or transferrin.

In some embodiments, non-viral expression vectors are introduced into the cells or tissues as naked DNA by direct injection or electroporation. Uptake efficiency of naked DNA may be improved by compaction or by using biodegradable latex beads. Such delivery may be improved further by treating the beads to increase hydrophobicity and thereby facilitate disruption of the endosome and release of the DNA into the cytoplasm.

### Viral Vectors

In some embodiments, the expression vector of the present application is a viral expression vector. In certain embodiments, viral expression vectors may be engineered to target certain diseases and cell populations by using the targeting characteristics inherent to the virus vector or engineered into the virus vector. Specific cells may be “targeted” for delivery of polynucleotides, as well as expression.

In some embodiments, the viral expression vector is selected from the group consisting of retroviral vectors, lentivirus vectors, adenovirus vectors, adeno-associated virus (AAV) vectors and herpes virus vectors.

In some embodiments, the viral expression vector is a lentivirus vector. In some embodiments, the lentivirus vector is a non-primate lentivirus vector, such as equine infectious anemia virus (EIAV).

In some embodiments, the viral expression vector comprises a mitogenic T cell-activating transmembrane protein and/or a cytokine-based T cell-activating transmembrane protein in the viral envelope. In some embodiments, the viral expression vector is a lentiviral vector comprising a mitogenic T cell-activating transmembrane protein and/or a cytokine-based T cell-activating transmembrane protein in the viral envelope.

In some embodiments, the viral expression vector is a recombinant AAV vector (rAAV). rAAVs can spread throughout CNS tissue following direct administration into the cerebrospinal fluid (CSF), e.g., via intrathecal and/or intracerebral injection. In some embodiments, rAAVs (such as AAV-9 and AAV-10) cross the blood-brain-barrier and achieve wide-spread distribution throughout CNS tissue of a subject following intravenous administration. In some cases, intravascular (e.g., intravenous) administration facilitates the use of larger volumes than other forms of administration (e.g., intrathecal, intracerebral). Thus, large doses of rAAVs (e.g., up to 1015 rAAV genome copies (GC)/subject) can be delivered at one time by intravascular (e.g., intravenous) administration. Methods for intravascular administration are well known in the art and include, for example, use of a hypodermic needle, peripheral cannula, central venous line, etc.

Any suitable AAV serotype may be utilized for the recombinant AAV, including but not limited to AAV1, AAV2, AAV3, AAV4, AAV5, AAV6, AAV7, AAV8, AAV9, AAV10, AAV11, AAV12, and pseudotyped combinations thereof. Pseudotyped (or chimeric) AAV vectors include portions from more than one serotype, for example, a portion of the capsid from one AAV serotype may be fused to a second portion of a different AAV serotype capsid, resulting in a vector encoding a pseudotyped AAV2/AAV5 capsid. Alternatively, the pseudotyped AAV vector may contain a capsid from one AAV serotype in the background structure of another AAV serotype. For example, a pseudotyped AAV vector may include a capsid from one serotype and inverted terminal repeats (ITRs) from another AAV serotype. Exemplary AAV vectors include recombinant pseudotyped AAV2/1, AAV2/2, AAV2/5, AAV2/7, AAV2/8 and AAV2/9 serotype vectors. Unless otherwise specified, the AAV ITRs, and other selected AAV components described herein, may be readily selected from among any AAV serotype, including, without limitation, AAV1, AAV2, AAV3, AAV4, AAV5, AAV6, AAV7, AAV8, AAV9, AAV10, AAV11, AAV12 or other known or as yet unknown AAV serotypes. These ITRs or other AAV components may be readily isolated from an AAV serotype using techniques available to those of skill in the art. In addition, AAV sequences may be isolated or obtained from academic, commercial, or public sources (e.g., the American Type Culture Collection, Manassas, Va.) or may be obtained through synthetic or other suitable means by reference to published sequences such as are available in the literature or in databases such as, e.g., GenBank, PubMed and the like.

It will be appreciated by those skilled in the art that the design of the expression vector of the present application can depend on such factors as the choice of the host cell to be transformed, the level of expression of protein desired, and the like.

mtON-Mitochondria

Another aspect of the present application relates to a mitochondrion (mtON-mitochondria) that comprises the mtON fusion protein of the present application in its inner membrane, wherein the mtON fusion protein is capable of pump protons from the mitochondrial matrix to the inner membrane space upon activation by light.

In some embodiments, the mtON-mitochondria are transferred directly into cells. In some embodiments, mtON-mitochondria are transferred into cells by first mixing them together with the cells followed by centrifugation. This method makes mitochondrial delivery possible into any cell type, and no additional incubation is required. The transfer efficiency remains high irrespective of the amounts of mitochondria used. In a specific embodiment, mtON-mitochondria are transferred into target cells via centrifugation at 1,500×g for 5 min without additional incubation. The exogenous mtON-mitochondria can be transferred regardless of cell type or species.

In a more specific embodiment, prior to mitochondrial transfer, recipient cells prelabelled with MitoTracker™ Green are harvested from culture flasks, and 1×10⁵ cells were transferred to a microcentrifuge tube. Cells were suspended in 100 μl of PBS and kept on ice for transfer. The mitochondrial suspension (in 10 μl of PBS) is added slowly to each tube of recipient cells suspended in 100 μl of PBS. The microcentrifuge tubes are centrifuged at 1,500×g for 5 min at 4° C. Cells are then rinsed twice with PBS and imaged or lysed for further testing.

In certain embodiments, mtON-mitochondria may be transferred to cells by use of Pep 1-conjugated mitochondria.

In certain embodiments, mtON-mitochondria may be transferred to cells by use of magnetic nanoparticles, such as by treating cultured cells with mitochondria labelled with anti-TOM22 magnetic beads and placing them on magnetic plates.

In certain embodiments, mtON-mitochondria may be transferred to cells by transferring mitochondria isolated from mesenchymal stem cells into cultured cancer cells. In specific embodiments, cancer cells are plated, mtON-mitochondria are added and cultures are centrifuged twice. Co-culture is then performed for 24 h to transfer mitochondria.

One of ordinary skill will understand that the means by which the mtON-mitochondria are transferred into target cells whether in vivo, ex vivo, or in vitro is not limiting on the scope of the application.

mtON Cells

Another aspect of the application relates to cells (mtON-cells) comprising mitochondria (mtON-mitochondria) comprising a mtON fusion protein. The mtON fusion protein comprises a first moiety that targets the fusion protein to the mitochondrial inner membrane and a second moiety that comprises a light-activated proton pump, wherein the first moiety orients the proton pump in the direction to pump protons from the mitochondrial matrix to the inner membrane space.

The cells types that may be targeted for use of mtON-mitochondria include, but are not limited to, T-cells, neurons, retinal cells, stem cells, hematopoietic stem cells, induced pluripotent stem cells, blood cells, epithelial cells, muscle cells, interneurons, glial cells, fat cells, hair follicles, keratinocytes.

In some embodiments, a mtON cell is generated by introducing an expression vector of the present application into a target cell with any conventional method, such as by naked DNA technique, cationic lipid-mediated transfection, polymer-mediated transfection, peptide-mediated transfection, virus-mediated infection, physical or chemical agents or treatments, electroporation, etc.

In some embodiments, a mtON cell is generated by transferring one or more mtON mitochondria into a target cell. In certain embodiments, the target cell is an animal cell. In certain embodiments, the target cell is a plant cell. In certain embodiments, the target cell is a human cell.

### Methods of Use

A further aspect of the present application relates to a method of treating, or ameliorating symptoms of, diseases or conditions that are characterized by lowered mitochondrial activity or mitochondrial dysfunction due to reduced PMF in a target cell population of a subject. In some embodiments, the method comprises the steps of introducing mtON fusion proteins into the mitochondria of the cell population to generate mtON cells, and exposing the mtON cells to light to activate the proton pump to increase mitochondrial activity, wherein the mtON fusion protein comprises a first moiety that targets the fusion protein to the mitochondrial inner membrane and a second moiety that comprises a light-activated proton pump, wherein the first moiety orients the proton pump in the direction to pump protons from the mitochondrial matrix to the inner membrane space.

In some embodiments, the method further comprises administering to the subject a second therapeutic agent. The second therapeutic agent can be administered to the subject before, after, or concurrently with the mtON fusion protein.

Diseases and conditions that are characterized by lowered mitochondrial activity include, but are not limited to, tumors, cancers, inflammatory disorders and immune disorders.

Diseases and conditions that are characterized by lowered mitochondrial activity also include disorders of mitochondrial metabolism, such as Kearns-Sayre syndrome (KSS), myoclonus epilepsy with ragged red fibers (MERRF), and mitochondrial encephalomyopathy, lactic acidosis and stroke-like episodes (MELAS).

Diseases and conditions that may be related mitochondrial dysfunction due to reduced PMF include, but are not limited to, ophthalmoplegia, retinal degeneration, heart block, myoclonus, ataxia, weakness, episodic vomiting, cerebral blindness, hemiparesis, hemianopsia and seizures.

The mtON fusion protein may be introduced into mitochondria of the target cell population by introducing a non-viral expression vector capable of expressing the mtON protein into the target cells. Alternatively, the mtON fusion protein may be introduced into the target cell population by infecting the target cells with a viral vector capable of expressing the mtON protein in the target cells. The location and timing of the mtON expression may vary depending on the target cell population and the diseases or conditions to be treated by the method.

In some embodiments, mtON cells are generated in vitro by transfecting or infecting cultured cells with mtON expression vectors. The culture mtON cells are then transferred into the subject for treating, or ameliorating symptoms of, diseases or conditions that are characterized by lowered mitochondrial activity or mitochondrial dysfunction.

In some embodiments, the cells are plant cells. In some embodiments, the cells are animal cells. In certain embodiments, the target cells are human cells.

In some embodiments, the cultured cells are cells autologous to the subject. In some embodiments, the cultured cells are cells allogeneic to the subject. Examples of such cells include, but are not limited to, T cells, natural killer cells, stem cells, hematopoietic stem cells, blood cells, neurons, interneurons, muscle cells, glial cells, fat cells, epithelial cells, hair follicles and karatinocytes.

After expression of the mtON protein, the resulting mtON cells will be exposed to light to activate the proton pump of the mtON protein. The wave length of the light depends on the characteristics of the light-sensitive proton pump on the mtON fusion protein; the general wavelength range for light-activated proton pumps and the wavelength range for each individual proton pump described in this application are the following: Arch and variants, 450-650 nm; Mac and variants, 425-625 nm; bacteriorhodopsin and variants, 400-650 nm; delta rhodopsin and variants, 400-650 nm.) The light intensity and length of light-exposure may be adjusted to establish protonmotive force (PMF) in mitochondria of the mtON cells to achieve desired therapeutic effect. In some embodiments, the activation light is provided by an LED system implanted in the subject. In some embodiments, the activation light is provided through an optical fiber.

Mitochondria supply energy for cellular activity. Mitochondria are much like batteries and use metabolic substrates to generate a protonmotive force, which is a charge separation that is used to do work. As we age, mitochondria become dysfunctional and the machinery that produces the protonmotive force becomes damaged resulting in impaired energy production. Since mitochondria are important for survival, dysfunction is implicated in numerous pathologies, such as androgenetic alopecia. Thus, an ideal approach is to selectively restore lost mitochondrial function in the hair follicle through a noninvasive approach amenable to home use. mtON can selectively generate a protonmotive force independent of mitochondrial function. As humans age mitochondrial function decreases. mtON can generate cellular energy in the absence of oxygen, metabolites, and a functioning electron transport chain. The delivery of mtON to hair follicle cells can stimulate hair growth on human tissue.

Hair forms a protective barrier and has roles in social interactions. Hair generation requires the activation of hair follicle stem cells through an energy intensive process and aging can alter this, resulting in the graying and thinning of hair. Androgenetic alopecia (male/female pattern hair loss) is a common form of hair loss and can result from stress, environmental insults or aging. There is no cure for baldness. Current therapies are limited by incomplete efficacy and serious adverse effects. In some embodiments, mtON is used to directly alter mitochondrial metabolism in hair follicles to reverse hair thinning and loss associated with aging.

Stress-induced hair graying occurs through reversible changes in mitochondrial function. Boosting mitochondrial function accelerates the hair growth cycle. In some embodiments, mtON is used to directly alter mitochondrial metabolism in hair follicles to reverse stress-induced hair graying.

In some embodiments, mtON tools may be adapted for expression in plants for myriad applications. In plants, mitochondria provide energy similarly to their role in metazoan organisms. Using mtON to manipulate protonmotive force in plants results in applications to control the growth rate of plants, crop yields, quality of crop yields, and disease or parasite resistance. Metabolism is a fundamentally important parameter for each of these aspects of plant life. Plants naturally use visible light for photosynthesis and are a suitable system to apply mtON. The absorbance spectrum of mtON is not widely used in plants and would allow the constructs to supplement plant energy. Of note, unlike respiration, mtON activity does not require oxygen or metabolic substrates. Thus, the use of mtON would enhance plant growth in combination with natural light-control of photosynthesis. This approach may reduce the amount of fertilizers needed for growth. Given the lack of oxygen required for mitochondrial function plants expressing mtON could offer opportunities for terraforming other planets or use in space, where oxygen and metabolites are limited.

In some embodiments, mtON may be adapted to weight control. Mitochondria are the metabolic hub of the cell and can signal energy status to the cell through signaling cascades. The activity of mtON can suppress and activate AMPK signaling activity, respectively. AMPK is a master regulator of a cells metabolic status and is activated under conditions of low energy or starvation. AMPK activity is suppressed under conditions of abundant energy sources or plentiful food. Using mtON tools it is possible to manipulate C. elegans feeding behavior. For example, the activation of mtON resulted in the worms behaving as if they were fed despite the lack of food. These studies also showed that this process is mediated through neurons only. Based on these findings mtON can be expressed to control hunger. The activation of mtON can alter behavior so humans would eat less in an effort to maintain a healthy weight.

In some embodiments, mtON tools may be adapted to grow cell-based meat, that is meat which is produced in a cell culture dish in the lab, which can potentially partially substitute animal meat for human consumption. Such a technique can be environmentally-friendly and reduce the need for slaughtering animals for meat. Cells can proliferate in vitro and eventually differentiate into muscles fibers, which can then be consumed. One rate-limiting factor for this technique is the relatively small yield of meat. Precursor cells can be genetically modified in vitro and express mtON. The mtON treatment can increase the rate of proliferation of the cells and ultimately increase the total yield of the cell-based meat. This process decreases the materials needed to grow the meat since energy would be provided in the form of light.

### Method of Use Relating to Hypoxia

In some embodiments, the present application provides a method for modulating hypoxia signaling in a subject. The method comprises the steps of: administering to the subject an effective amount of an expression vector comprising a polynucleotide encoding a mtON fusion protein, expressing the mtON fusion protein in a group of target cells to generate mtON cells; and exposing the mtON cells to light for a desired period of time to modulate hypoxia signaling.

In some embodiments, the present application provides a method for improving hypoxia resistance in a subject. The method comprises the steps of: administering to the subject an effective amount of an expression vector comprising a polynucleotide encoding a mtON fusion protein, expressing the mtON fusion protein in a group of target cells to generate mtON cells; and exposing the mtON cells to light for a desired period of time to improve hypoxia resistance.

In some embodiments, the present application provides a method for preventing/ameliorating ischemia reperfusion injury in a subject. The method comprises the steps of: administering to the subject an effective amount of an expression vector comprising a polynucleotide encoding a mtON fusion protein, expressing the mtON fusion protein in a group of target cells to generate mtON cells; and exposing the mtON cells to light for a desired period of time to prevent or ameliorate ischemia reperfusion injury. In some embodiments, the ischemia reperfusion injury is caused by heart attack or stroke. In some embodiments, the target cells are cardiomyocytes, smooth muscle cells, cardiac neurons and/or endothelial cells. In some embodiments, the expression vectors are administered prior to the start of ischemia. In some embodiments, the expression vectors are administered during ischemia. In some embodiments, the expression vectors are administered after ischemia.

In some embodiment, the present application provides a method to preventing/ameliorating ischemia reperfusion injury in a subject in the context of elective surgery or organ transplant, transfection or other suitable mechanisms may introduce the expression vector to the subject as a way of preparing the subject to modulate or improve hypoxia resistance. In certain embodiments, mtON fusion proteins are introduced into cardiac muscle during an elective cardiac surgery when the heart is stopped or put on bypass. In certain embodiments, mtON fusion proteins are introduced into transplanted tissues/organ to prevent or ameliorate ischemia reperfusion injury to the transplanted tissues/organ.

### Method of Use Relating to Aging and Lifespan

Without being bound by theory, deregulation of mitochondrial function is causally linked with the progression of aging. By bypassing upstream chemical reaction aimed to generate the proton gradient (for example the activity of the TCA cycle) needed for ATP synthesis, external light energy is harnessed to directly increase the proton gradient. Through this process lifespan of cells is extended and revitalized. As described herein, using mtON combined with treatment with light and ATR during adulthood, could significantly ameliorate age-associated dysfunction and extend the healthy lifespan of a genetic model organism. mtON is a tool which is widely applicable to other organisms and represents a novel concept of cell lifespan extension and revitalization.

In some embodiments, the mtON fusion protein is used to treat aging and related maladies in a subject by delaying the progression of normal aging by replacing the need to use tricarboxylic acid (TCA) (and/or Oxidative respiratory chain) activity. In certain embodiments, the mtON fusion protein is used to extend lifespan of a cell or a subject through the conversion of external light energy into generating pH gradient that is needed for ATP synthesis. In some embodiments, the mtON fusion protein is used to ameliorate age-associated physical activity decline in a subject.

As such, the presently application provides methods for treat aging and related maladies in mammals such as humans, as well as those mammals of importance due to being endangered, such as Siberian tigers; of economic importance, such as animals raised on farms for consumption by humans; and/or animals of social importance to humans, such as animals kept as pets or in zoos.

In some embodiments, the present application provides a method for revitalizing cells in a subject. The method comprises the steps of expressing mtON fusion proteins in a target cell population to form mtON cells, wherein the mtON fusion protein comprises a first moiety that targets the fusion protein to the mitochondrial inner membrane and a second moiety that comprises a light-activated proton pump, wherein the first moiety orients the proton pump in the direction to pump protons from the mitochondrial matrix to the inner membrane space; and exposing the mtON cells to light to activate the proton pump and revitalize the mtON cells.

In certain embodiments, the target cells are selected from the group consisting of stem cells, epithelial cells, muscles cells (such as enhancing recovery of athletes' from injury/shortening the recovery time), sperm cells (such as enhancing motility of spem cells), retinal cells, T-cells, and hematopoietic stem cells and blood cells.

In some embodiments, the target cells are retinal cells and the mtON fusion proteins are introduced into the retinal cells with AAV mediated gene transfer to the eye; for example, to treat eye diseases.

In some embodiments, the target cells are blood cells. In some embodiments, the blood cells are isolated from the subject, infected or transfected in vitro with a mtON expression vector, and then transferred back to the subject.

In some embodiments, the target cells are stem cells. In some embodiments, the stem cells are isolated from the subject, differentiated in vitro, infected or transfected with a mtON expression vector in vitro, and then transferred back to the subject.

In some embodiments, the present application provides a method for ameliorating age-associated skin damage in a subject. The method comprises the steps of administering to the subject an effective amount of an expression vector carrying the coding sequence of a mtON fusion protein, wherein the mtON fusion protein comprises a first moiety that targets the fusion protein to the mitochondrial inner membrane and a second moiety that comprises a light-activated proton pump, wherein the first moiety orients the proton pump in the direction to pump protons from the mitochondrial matrix to the inner membrane space, expressing the mtON fusion protein in skin cells of the subject to generate mtON skin cells, and exposing the mtON skin cells to light for a desired period of time to ameliorating age-associated skin damage in the mtON skin cells. In some embodiments, the expression vector is administered topically.

In some embodiments, the mtON fusion protein, the mtON expression vector and/or the mtON cells may be formulated in a topical treatment composition. In some embodiments, the mtON fusion protein, the mtON expression vector and/or the mtON cells may be co-formulated with one or more skincare ingredients. In some embodiments, the one or more skincare ingredients may be small molecule compounds, polymers, peptides or cells. In some embodiments, the one or more skincare ingredients are selected from the group of alpha-hydroxy acids, polyhydroxy acids, beta-hydroxy acid (salicylic acid), hydroquinone, kojic acid, retinoids, L-ascorbic acid, hyaluronic acid, copper peptide, alpha-lipoic acid, and DMAE (dimethylaminoethanol),

In some embodiments, the topical treatment composition is formulated for application to human skin. More specifically, the formulation can be configured to penetrate topically from the epidermis to the dermis. In some embodiments, the formulation can be configured to penetrate topically through the epidermis and dermis layers. In some embodiments, the formulation can be configured to penetrate topically through the epidermis layer and have low penetration into the dermis layer. Often, the penetration of a component in a formulation may be assessed using various permeation studies, including but not limited to those using a Franz diffusion cell. In some embodiments, the formulation comprises a carrier, a microsphere, a liposome, or a micelle in order to carry the mtON fusion protein, the mtON expression vector and/or the mtON cells and control the release time and/or penetration depth of the mtON fusion protein, the mtON expression vector and/or the mtON cells through the skin. In some cases, a formulation herein is a cream, an ointment, a gel, a liquid, an oil, a powder, a lotion, a serum, an emulsion, a moisturizer, a foam, a face mask, a mousse, an aerosol, a spray, a cleanser, a toner, a topical patch, a hydrogel patch, or a shampoo.

In some embodiments, the formulation further comprises a therapeutic, nutraceutical, or cosmetic excipient. In some embodiments, the administering comprises applying the formulation to a portion of the skin of the subject. In some embodiments, the formulation extends a lifespan of a plurality of cells of the subject, induces SIRT6 expression in a plurality of cells of the subject, increases cell renewal rates in a plurality of cells of the subject, promotes apoptosis in a plurality of cells of the subject, promotes DNA repair in a plurality of cells of the subject, increases collagen production in a plurality of cells of the subject, increases hyaluronic synthase production in a plurality of cells of the subject, decreases ATRX nuclear foci accumulation in a plurality of cells of the subject, decreases p16 expression in a plurality of cells of the subject, decreases senescence associated beta-galactosidase production in a plurality of cells of the subject, decreases IL8 expression in a plurality of cells of the subject, decreases MMP1 expression in a plurality of cells of the subject, increases BLM expression in a plurality of cells of the subject, and/or prevents UV-induced DNA damage in a plurality of cells of the subject.

In particular embodiments, a mtON fusion protein, mtON expression vector and/or a mtON cell composition can be formulated for topical application. For example, the composition may be formulated for application onto skin. In some embodiments, the composition is configured as a topical supplement. Formulations such as those for topical application can be a cream, an ointment, a gel, a liquid, a powder, a lotion, a serum, an emulsion, a moisturizer, a foam, a face mask, a mousse, an aerosol, a spray, a cleanser, a toner, a topical patch, a hydrogel patch, or a shampoo. mtON fusion protein, mtON expression vector and/or mtON cells applied topically can be applied to an affected area, to an area which may become affected in the future, a portion of the subject, or substantially the entire subject. In some cases, a topical treatment can be applied with a buffer, another topical treatment, a cream, or a moisturizer.

A composition, such as for topical application, can be formulated as a cosmetic composition. Examples of cosmetic compositions can include makeup, foundation, sunscreen, after sun lotion, and skin care products, including anti-aging skin care products. In some cases, makeup compositions can leave color on the face, and can include foundation, bronzer, mascara, concealer, eye liner, brow color, eye shadow, blusher, lip color, powder, a solid emulsion compact, or other makeup items. In some cases, skin care products can be those used to treat or care for, or somehow moisturize, improve, accelerate renewal, protect, prevent damage, or clean the skin. A skin-care product can be applied as a cream, a topical patch, a hydrogel patch, a transdermal patch, an ointment, a gel, a liquid, a powder, a lotion, a serum, an emulsion, an oil, a clay, a moisturizer, a foam, a face mask, a mousse, an aerosol, a spray, a cleanser, a toner, or a shampoo. In some cases, skin-care products can be in the form of an adhesive, a bandage, exfoliant, a toothpaste, a moisturizer, a lotion, a primer, a lipstick, a lip balm, an anhydrous occlusive moisturizer, an antiperspirant, a deodorant, a personal cleansing product, an occlusive drug delivery patch, a nail polish, a powder, a tissue, a wipe, a hair conditioner, or a shaving cream.

In some cases, a composition can comprise a skin conditioning agent (e.g., a humectant, exfoliant, emollient, or hydrator). A humectant can be for moisturizing, reducing scaling, or stimulating removal of built-up scale from the skin. An exfoliant can be for the removal of old skin cells from the surface, and can be a physical exfoliant or a chemical exfoliant. An emollient can be a preparation or ingredient which can soften dry, rough, or flakey skin. A hydrator can be for moisturizing, reducing scaling, or stimulating removal of built-up scale from the skin. In some cases, emollient is an agent that prevents water loss and has a softening and soothing effect on skin. In some embodiments, emollients may comprise at least one of plant oils, mineral oil, shea butter, cocoa butter, petrolatum, fatty acids (animal oils, including emu, mink, and lanolin), triglycerides, benzoates, myristates, palmitates, stearates, glycolipids, phospholipids, squalene, glycerin, rose hip oil, andiroba oil, grape seed oil, avocado oil, plum seed oil, pracaxi oil, Calycophyllum spruceanum oil, almond oil, argan oil, caprylic/capric triglyceride, jojoba butter, jojoba oil, Spectrastat G2, ceramide, and algae extract. In some cases, the composition comprises a skin hydrating agent, also referred to as a skin hydrator. In some cases, the skin hydrating agent include but are not limited to glycerin, squalene, sorbitol, hyaluronic acid, hyaluronic acid derivatives, sodium hyaluronate, sodium hyaluronate crosspolymer, niacinamide, glycoproteins, pyrrolidone carboxylic acid (PCA), lysine HCl, allantoin and algae extract. In some embodiments, the composition comprises at least 1%, 2%, 3%, 4%, 5%, 6%, 7%, 8%, 9%, or 10% skin conditioning agent. In some embodiments, the composition comprises about 1% to about 70%, about 1% to about 60%, about 1% to about 50%, about 5% to about 50%, about 5% to 45%, or about 5% to 40% skin conditioning agent.

A composition can comprise a shine control agent, which can improve or regulate the shiny appearance of skin. Shine control agents can be porous in nature. Such agents can provide a reservoir to absorb excess moisture to reduce the appearance of shine. Shine control agents can be silicas, magnesium aluminum silicates, talc, sericite and various organic copolymers. Particularly effective shine control agents can include silicates or carbonates that are formed by reaction of a carbonate or silicate with the alkali (IA) metals, alkaline earth (IA) metals, or transition metals, and silicas (silicon dioxide). Preferred shine control agents are selected from the group consisting of calcium silicates, amorphous silicas, calcium carbonates, magnesium carbonates, zinc carbonates, bentonite clay, and combinations thereof.

A composition can comprise a film forming agent, which can aid film substantivity and adhesion to the skin. A film forming agent can improve long wear and non-transfer performance of a composition. Film forming agents can be water soluble, water insoluble, or water dispersing. Film forming agents can be 1) organic silicone resins, fluorinated silicone resins, copolymers of organic silicone resins, trimethylsiloxysilicate, GE's copolymers of silicone resins, SF1318 (silicone resin and an organic ester of isostearic acid copolymer) and CF1301 (silicone resin and alpha methyl styrene copolymer), Dow Corning's pressure sensitive adhesives copolymers of silicone resins and various PDMS's (BIO-PSA series); and 2) acrylic and methacrylic polymers and resins, silicone-acrylate type copolymers and fluorinated versions of, including silicones plus polymer from 3M, KP545 from Shin-Etsu, alkyl-acrylate copolymers, KP 561 and 562 from Shin-Etsu; 3) decene/butene copolymer from Collaborative Labs; 4) polyvinyl based materials, PVP, PVPNA, including Antaron/Ganex from ISP (PVP/Triacontene copolymer), Luviskol materials from BASF; polyurethanes, the Polyderm series from Alzo including but not limited to Polyderm PE/PA, Polyderm PPI—SI-WS, Polyderm PPI-GH, Luviset P.U.R. from BASF; 6) polyquaternium materials, Luviquat series from BASF; 7) acrylates copolymers and acrylates/acrylamide copolymers, Luvimer and Ultrahold series, both available from BASF; 8) styrene based materials; and 9) chitosan and chitosan based materials including cellulose and cellulose-based materials.

A composition can comprise a thickening agent or an emulsifying agent. A thickening agent may be used to increase the viscosity of liquid base materials to be used in a cosmetic composition. The selection of a particular thickening agent can depend on a type of composition desired (e.g., gel, cream, lotion, or wax based), the desired rheology, the liquid base material used, and other materials to be used in the composition. Examples of thickening agent or an emulsifying agent can include waxy materials such as candelilla, carnauba waxes, beeswax, spermaceti, carnauba, baysberry, montan, ozokerite, ceresin, paraffin, synthetic waxes such as Fisher-Tropsch waxes, silicone waxes (DC 2503 from Dow Corning), microcrystalline waxes and the like; soaps, such as the sodium and potassium salts of higher fatty acids, acids having from 12 to 22 carbon atoms; amides of higher fatty acids; higher fatty acid amides of alkylolamines; dibenzaldehyde-monosorbitol acetals; alkali metal and alkaline earth metal salts of the acetates, propionates and lactates; and mixtures thereof. Also useful are polymeric materials such as, locust bean gum, sodium alginate, sodium caseinate, egg albumin, gelatin agar, carrageenin gum sodium alginate, xanthan gum, quince seed extract, tragacanth gum, starch, chemically modified starches and the like, semi-synthetic polymeric materials such as cellulose, cellulose derivatives, cellulose ethers hydroxyethyl cellulose, methyl cellulose, hydroxypropyl cellulose, carboxymethyl cellulose, hydroxy propylmethyl cellulose, polyvinylpyrrolidone, polyvinylalcohol, guar gum, hydroxypropyl guar gum, soluble starch, cationic celluloses, cationic guars and the like and synthetic polymeric materials such as carboxyvinyl polymers, polyvinylpyrrolidone, polyvinyl alcohol polyacrylic acid polymers, poly(acrylic acid), carbomers, polymethacrylic acid polymers, polyvinyl acetate polymers, polyvinyl chloride polymers, polyvinylidene chloride polymers and the like. Inorganic thickeners may also be used such as aluminum silicates, such as, for example, bentonites, or a mixture of polyethylene glycol and polyethylene glycol stearate or distearate. An emulsifier may be used to help keep hydrophilic and hydrophobic ingredients from separating in an emulsion. In some cases, emulsifiers include but are not limited to Olivem, Oliwax LC, polysorbates, laureth-4, and potassium cetyl sulfate.

A cosmetic composition can provide a temporary change in an appearance or can provide a long-term change in an appearance. In some cases, a cosmetic composition can be formulated to provide a short-term change in an appearance (e.g., color deposition or plumping of skin) as well as a long-term change in appearance (e.g., reduction in spots, appearance of fine lines, appearance of wrinkles, or other features which can affect appearance).

A composition can comprise an additive that has an additive or synergistic effect when applied with the mtON fusion protein, the mtON expression vector and/or the mtON cells as disclosed herein. For example, a composition comprising the mtON fusion protein, the mtON expression vector and/or the mtON cells and an additive can have a greater effect on senescence, and age-related disease or condition, or an age-associated disorder (e.g., delay the onset of, reduce the occurrence of, or ameliorate one or more symptoms) than the individual effect of the additive, the polypeptide, or the sum of the individual effects of the additive and the mtON fusion protein, the mtON expression vector and/or the mtON cells. Additives can be a polypeptide, a glycosaminoglycan, a carbohydrate, a polyphenol, a protein, a lipid, a plant aqueous or oil extract, a nucleic acid, an antibody, a small molecule, a vitamin, a humectant, an emollient, or another suitable additive. In some embodiments, the composition comprises a UV blocker. In some embodiments, the UV blocker may include but is not limited to aminobenzoic acid, avobenzone, cinoxate, dioxybenzone, homosalate, meradimate, octocrylene, octinoxate, octisalate, oxybenzone, padimate O, ensulizole, sulisobenzone, titanium dioxide, trolamine salicylate, and zinc oxide.

Often the methods, systems, and compositions provided herein comprise a vitamin. In some instances, the vitamin provides skin soothing, skin restoring, skin replenishing, and/or hydrating effects. In some instances, the vitamin provides antioxidant effects. In some instances, the vitamin acts as an emollient. In some instances, the vitamin improves the appearance of enlarged pores, uneven skin tone, fine lines, dullness, and/or a weakened skin surface. In some instances, the vitamin is vitamin A, vitamin D, vitamin E, vitamin F, vitamin K, vitamin B1 (thiamine), vitamin B2 (riboflavin), vitamin B3 (niacin), vitamin B5 (pantothenic acid), vitamin B7 (biotin), vitamin B6, vitamin B12 (cyanocobalamin), vitamin B9, folic acid, niacinamide, and mixtures thereof. In some instances, the composition comprises a derivative of a vitamin. In some instances, a derivative of a vitamin is used to improve stability of the vitamin in the composition and/or compatibility of the vitamin derivative with other ingredients in the composition. In some instances, the composition comprises vitamin B3 or its derivative and vitamin E or its derivative. In some instances, the composition comprises niacinamide and vitamin E or its derivative. In some instances, the composition comprises vitamin C or its derivative, vitamin B3 or its derivative, and vitamin E or its derivative. In some embodiments, the composition comprises at least 0.01%, 0.05%, 0.5%, 1%, 2%, 3%, 4%, 5%, 6%, 7%, 8%, 9%, or 10% vitamin. In some embodiments, the composition comprises about 0.1% to about 10%, about 0.1% to about 5%, about 0.5% to about 10%, about 0.5% to about 5%, about 1% to 10%, or about 1% to 5% vitamin.

If intended for transdermal administration, the composition may include a transdermal patch or iontophoresis device. In some cases, biodegradable microspheres (e.g., polylactic acid) may also be employed as carriers for a composition. In some cases, the transdermal patch is prepared to deliver the formulation to the epidermal layer of the skin. In some cases, the transdermal patch is prepared to deliver the formulation to the epidermal and dermal layers of the skin. In some cases, the formulation is prepared as to be minimally delivered systemically in the subject or is not intended to be delivered directly into the bloodstream of the subject.

In some cases, the age-related disease or condition or age-associated disorder can be a disease, condition, or disorder affecting the skin, such as a skin disorder or a dermatosis, which can comprise wrinkles, lines, dryness, itchiness, spots, age spots, bedsores, ulcers, cancer, dyspigmentation, infection (e.g., fungal infection), or a reduction in a skin property such as clarity, texture, elasticity, color, tone, pliability, firmness, tightness, smoothness, thickness, radiance, luminescence, hydration, water retention, skin barrier, evenness, laxity, or oiliness, or other dermatoses. In some instances, the age-related disease or condition or age-associated disorder is hyperpigmentation of the skin. In some instances, the hyperpigmentation disorder is melasma, age spots, lentigines, and/or progressive pigmentary purpura. In some instances, the hyperpigmentation is a result of sun damage, inflammation, hormone changes, or skin injuries. In some instances, the hyperpigmentation occurs after a cosmetic procedure, including but not limited to a laser treatment, a light treatment, or a chemical peel; administration of an antibiotic, an oral contraceptive, or a photosensitizing drug; or application of a topical agent. In some instances, the hyperpigmentation is a result of excess production of melanin.

In some instances, treatment of the age-related disease or condition or age-associated disorder with the methods, systems and compositions disclosed herein results in lightening, increasing luminescence, brightening, evening, smoothing and/or firming of the skin's appearance. In some instances, treatment with the methods, systems, and compositions disclosed herein improves the epidermal barrier, skin hydration level, skin water retention, appearance of wrinkles, smoothness, firmness, elasticity, appearance of radiance and luminosity, and/or improves or maintains the ceramide level in the skin. In some instances, the effect of treatment with the methods, systems, and compositions disclosed herein is assessed by measuring skin moisture content, trans-epidermal water loss (TEWL), dermal thickness and echogenicity, intracutaneous analysis, skin viscoelastic properties, or skin surface profile. In some instances, the effect of treatment with the methods, systems, and compositions disclosed herein assesses for reduction in appearance of lines/wrinkles, appearance of skin tone (evenness), appearance of pores, appearance of texture/smoothness, firmness (visual), elasticity (tactile), epidermal barrier, skin roughness, skin hyperpigmentation, or overall appearance. In some instances, the effect of treatment with the methods, systems, and compositions disclosed herein is measured using an instrument, including but not limited to a corneometer for measuring skin moisture content/hydration, a VapoMeter for measuring the trans-epidermal water loss (TEWL), an ultrasound measuring dermal thickness (density) and echogenicity, a non-invasive optical skin imaging instrument for measuring skin evenness and chromophore mapping, a cutometer using suction for measuring viscoelastic properties of the skin (firmness and elasticity), skin profilometry, multi-spectral analysis, and colorimetry for measuring skin surface profile, lines, and wrinkles.

In some instances, the methods, systems, and compositions provided herein may reduce hyperpigmentation of the skin. In some instances, hyperpigmentation is associated with excess production of melanin. In some instances, the methods, systems, and compositions provided herein reduces the excess production of melanin. In some instances, the methods, systems, and compositions provided herein reduce the presence of melanin pigment in the skin. In some instances, the methods, systems, and compositions provided herein reduce the expression levels of proteins involved in melanogenesis, including tyrosinase, melanocyte inducing transcription factor (MITF) and dopachrome tautomerase (DCT), by the cells in the treated skin. In some instances, the methods, systems, and compositions provided herein result in reduction of tyrosinase activity, reduction of the expression or activation of tyrosinase, scavenging of the intermediate products of melanin synthesis, reducing the transfer of melanosomes to keratinocytes, reduction of existing melanin content, or reduction in melanocyte activity or viability.

An age-related disease or condition or age-associated disorder can be caused by UV damage, DNA damage, ATRX foci accumulation in cell nuclei, increased p16 expression, increased senescence-associated.beta.-galactosidase activity, accumulation of senescent cells in the tissue, increased SASP production, chemically induced senescence, chronological aging, decreased hyaluronic acid production, decreased expression of sirtuin 6, altered insulin-like growth factor-1 (IGF-I) pathway signaling, increased production of matrix metallopeptidase 1 (MMP1), thin epidermal layer of the skin, or genetic variants. In some instances, the age-related disease or condition or age-associated disorder is initiated or exacerbated by a therapeutic regimen, for example, a side effect of a therapeutic drug. An age-related disease or condition or age-associated disorder can affect the health or appearance of skin directly or indirectly. Topical application of a mtON fusion protein, a mtON expression vector and/or mtON cells can improve the health or appearance of skin in some such cases.

An age-related disease or condition or age-associated disorder can comprise a cell proliferative disorder. A cell-proliferative disorder can affect the health or appearance of the skin. In some cases, a treatment administered for a cell-proliferative disorder, such as chemotherapy or radiation can affect the health or appearance of the skin. Topical application of a mtON fusion protein, a mtON expression vector and/or mtON cells can improve the health or appearance of skin in some such cases.

Also provided herein are methods for treating the skin of a subject comprising administering to a subject a composition that can promote a decrease in a number of senescent cells in a tissue or organism, inducing a pro-apoptotic state in the treated cells, inducing SIRT6 expression, preventing DNA-induced senescence, and/or enhancing DNA repair capacity. In some cases, a skin disease such as a dermatological disease or condition can comprise skin sagging or wrinkling, accumulation of senescent cells in the tissue, decreased epidermal thickness, decreased collagen production, increased MMP-1 production, decreased DNA repair capacity, decreased SIRT6 expression, skin disorganization, a thin epidermal layer of the skin, inflammation, a senescence-associated secretory phenotype, or stem cell exhaustion of the skin.

### Method of Use Relating to Cancer Treatment

The fact that both cancer and T cells share the same type of metabolic process in a tumor microenvironment is an important obstacle for developing effective immunotherapies. As the demand for both oxygen and glucose in the niche is extremely high, tumor-infiltrating T cells, such as Car-T cells, face fierce metabolic competition from the target tumor/cancer cells. Without being bound by theory, expression of the mtON fusion protein in T cells may promote T cell metabolism and overcome local suppression/competition by tumor/cancer cells and enhance the cancer killing effect of the T cells.

In some embodiments, the present application is a method of treating cancer in a subject. The method comprises the steps of: expressing mtON fusion proteins in Car-T cells to generate mtON Car-T cells, wherein the mtON fusion protein comprises a first moiety that targets the fusion protein to the mitochondrial inner membrane and a second moiety that comprises a light-activated proton pump, wherein the first moiety orients the proton pump in the direction to pump protons from the mitochondrial matrix to the inner membrane space; infusing the mtON Car-T cells into the subject, and exposing the mtOn Car-T cells to light to enhance the tumor/cancer suppression activity of the mtON Car-T cells in the subject. Examples of cancer include, but are not limited to, carcinoma, melanoma, sarcoma, lymphoma, leukemia, germ cell tumor, and blastoma.

In some embodiments, mtON is expressed in target Car-T cells by infecting the Car-T cells with a lentiviral expression vector that comprises the coding sequence of the mtON fusion protein.

### Method of Use Relating to Treatment of Other Conditions

In some embodiments, the present application provides a method for treating acute lung injury by delivering mtON cells to aveolar epithelia by intranasal instillation.

In some embodiments, in situ blood perfused regional ischemia is treated by targeting myocardial cells through injection of mtON-mitochondria-containing buffer which leads to decreased necrosis and enhanced post-ischemic function. In some embodiments, transient focal cerebral ischemia is treated by targeting pre-infarct cortex cells by direct injection or autologous secretions of mtON-mitochondria which promotes adjacent neuronal survival and plasticity after injury transfer.

In some embodiments, Parkinson's disease is treated by targeting brain neurons by local injection at medial forebrain bundle of mtON-mitochondria which leads to improved locomotive activity and attenuated deterioration of dopaminergic neurons. In particular embodiments, Parkinson's disease is prevented by targeting brain neurons by local injection at medial forebrain bundle of mtON-mitochondria which leads to improved locomotive activity and attenuated deterioration of dopaminergic neurons, cells that dies are highly metabolically active, so mtON prevents them from reaching high metabolic toxicity.

In certain embodiments, acute myocardial infarction is treated by targeting myocardial cells by intravenous injection of mtON-mitochondria which leads to increased ETC activity, decreased ROS formation, apoptosis and necrosis.

In some embodiments, spinal cord injury (L1/L2 contusion) is treated by targeting brain macrophages, endothelium, pericytes, glia through microinjection at mediolateral grey matter which leads to maintenance of acute mitochondrial bioenergetics and enhanced behavioral recovery.

In some embodiments, non-alcoholic fatty liver disease is treated by targeting multiple tissues by intravenous injection mtON-mitochondria which leads to decreased lipid content and restored cellular redox balance. In certain embodiments, acetaminophen-induced liver injury is treated by targeting multiple tissues through intravenous injection mtON-mitochondria which leads to increased hepatocytes energy supply, reduced oxidation stress.

### Method of Use for Improving Farm Animal, Including Fish, Efficiency

Without being bound by theory, feed efficiency (FE) remains an important trait for commercial breeding companies because feed represents 50 to 70% of the cost of raising a bird to market weight. Genetic selection for FE has been responsible for more than 80% of the improvement in feed efficiency in modern broilers. Compared with high FE mitochondria, mitochondria obtained from low FE broilers appear to exhibit decreased electron transport chain coupling, increased electron leak with subsequent increased reactive oxygen species (ROS) production, increased protein oxidation, and lower respiratory chain complex activities. Thus, improvements in mitochondrial function provided by use of mtON in low FE animals can increase FE. mtON use results in less feeding of farm animals or fish.

In some embodiments, the present application provides a method for improving farm animal, including fish, efficiency. The method comprises the step of expressing the mtON fusion protein in cells of a farm animal and exposing mtON expressing cells to lights.

In some embodiments, mtON fusion protein is used for a method to improve animal breeding. In some embodiments, mtON fusion protein is used to treat gametes used in animal fertility treatments. In some embodiments, mtON fusion protein is used to treat sperm in in vitro fertilization of farm animals.

Administration of mtON Expression Vectors

Any suitable route or mode of administration can be employed for providing a subject with a therapeutically or prophylactically effective dose of the mtON expression vector. Exemplary routes or modes of administration include parenteral (e.g., intravenous, intraarterial, intramuscular, subcutaneous, intratumoral), topical (nasal, transdermal, intradermal or intraocular), mucosal (e.g., nasal, sublingual, buccal, rectal, vaginal), inhalation, intralymphatic, intraspinal, intracranial, intraperitoneal, intratracheal, intravesical, intrathecal, enteral, intrapulmonary, intralymphatic, intracavital, intraorbital, intracapsular and transurethral, as well as local delivery by catheter or stent.

### Pharmaceutical Compositions

A pharmaceutical composition comprising a mtON expression vector in accordance with the present disclosure may be formulated in any pharmaceutically acceptable carrier(s) or excipient(s).

In some embodiments, mtON expression vectors can be incorporated into a pharmaceutical composition suitable for parenteral administration. In some embodiments, the pharmaceutical composition comprises a buffer. Suitable buffers include but are not limited to, sodium succinate, sodium citrate, sodium phosphate or potassium phosphate. In some embodiments, the pharmaceutical composition comprises sodium chloride at a concentration of 0-300 mM (optimally 150 mM for a liquid dosage form).

In some embodiments, the pharmaceutical composition is in a lyophilized dosage form and comprise a cryoprotectant. Examples of cryoprotectants include, but are not limited to, sucrose (optimally 0.5-1.0%), trehalose and lactose. In some embodiments, the pharmaceutical composition further comprises a bulking agent. Examples of bulking agents include, but are not limited to, mannitol, glycine and arginine.

Therapeutic preparations can be lyophilized and stored as sterile powders, preferably under vacuum, and then reconstituted in bacteriostatic water (containing, for example, benzyl alcohol preservative) or in sterile water prior to injection. Pharmaceutical composition may be formulated for parenteral administration by injection e.g., by bolus injection or continuous infusion.

The pharmaceutical forms suitable for injectable use include sterile aqueous solutions or dispersions and sterile powders for the extemporaneous preparation of sterile injectable solutions or dispersions. The form should be sterile and fluid to the extent that easy syringability exists. It should be stable under the conditions of manufacture and storage and should be preserved against the contaminating action of microorganisms, such as bacteria and fungi. The pharmaceutical carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants.

Sterile injectable solutions can be prepared by incorporating the composition in the required amount in the appropriate solvent with various of the other ingredients enumerated above, followed by filtered sterilization. Generally, dispersions can be prepared by incorporating the various sterilized active ingredient into a sterile vehicle containing the basic dispersion medium and the required other ingredients from those enumerated above. In the case of sterile powders for the preparation of sterile injectable solutions, methods of preparation include vacuum drying and freeze drying techniques, which yield a powder of the active ingredient plus any additional desired ingredient from previously sterile filtered solution thereof.

Parenteral compositions may be formulated in dosage-unit form for ease of administration and uniformity of dosage. Dosage-unit form, as used herein, refers to physically discrete units suited as unitary dosages for the subjects to be treated, each unit containing a predetermined quantity of active material calculated to produce the desired therapeutic effect in association with the required pharmaceutical carrier. The specification for the dosage-unit forms of the present application can be chosen based upon: (a) the unique characteristics of the active material and the particular therapeutic effect to be achieved, and (b) the limitations inherent in the art of compounding such an active material for the treatment of conditions in living subjects having a condition in which bodily health is impaired as described herein.

An effective amount of a composition disclosed herein is a nontoxic, but sufficient amount of the composition, such that the desired prophylactic or therapeutic effect is produced. The exact amount of the composition that is required will vary from subject to subject, depending on the species, age, condition of the animal, severity of the inflammation or tumor-related disorder in the animal, the particular carrier or adjuvant being used, its mode of administration, and the like. Accordingly, the effective amount of any particular therapeutic composition disclosed herein will vary based on the particular circumstances, and an appropriate effective amount can be determined in each case of application by one of ordinary skill in the art using only routine experimentation.

The present application is further illustrated by the following examples that should not be construed as limiting. The contents of all references, patents, and published patent applications cited throughout this application, as well as the Figures and Tables, are incorporated herein by reference.

### EXAMPLES

### Example 1: Materials and Methods

Construction of mtON Fusion Protein

mtON was constructed by fusing the 187 N-terminal amino acids of the mouse IMMT1 protein (SEQ ID NO:6) to the light-activated proton pump (Mac) from Leptosphaeria maculans (SEQ ID NO:10), resulting in the novel fusion construct, Immt1::Mac, referred to as mtON (SEQ ID NO:11) (see FIG. 1). mtON is commonly fused with a fluorescent protein to meet experimental needs. The Immt1 coding sequence was amplified by PCR from mouse cDNA and Mac coding sequence was amplified from plasmid DNA pFCK-Mac-GFP (Addgene plasmid #22223).

The light-activated proton pump from Leptosphaeria maculans (Mac) fused to eGFP was amplified from plasmid DNA pFCK-Mac-GFP, (Addgene plasmid #22223) (forward amplification primer: ACACCTGCAGGCTTGATCGTGGACCAGTTCGA (SEQ ID NO:13), reverse amplification primer: CACAGCGGCCGCTTACTTGTACAGCTCGTCCA (SEQ ID NO: 14)).

The N-terminal 187 amino acids of the Immt1 gene were amplified by PCR from mouse cDNA (forward amplification primer: ACAACCGGTAAAAATGCTGCGGGCCTGTCAGTT (SEQ ID NO:15), reverse amplification primer: CACCCTGCAGGTTCCTCTGTGGTTTCAGACG (SEQ ID NO:16)).

The ubiquitously expressed gene promoter Peft-3 (also known as Peef-1A.1) was amplified by PCR from pDD162 (forward amplification primer: AACAAAGCTTGCACCTTTGGTCTTTTA (SEQ ID NO:17), reverse amplification primer: ACATCTAGAGAGCAAAGTGTTTCCCA (SEQ ID NO:18)).

The body wall muscle promoter Pmyo-3 was PCR amplified from pDJ16 (forward amplification primer: ACAGCTAGCTGTGTGTGATTGCT (SEQ ID NO:19), reverse amplification primer: ACAACCGGTGCGGCAATTCTAGATGG (SEQ ID NO:20)). PCR fragments were ligated into pFH6.II (pPD95.81 with a modified multi-cloning site) for C. elegans expression using restriction digest cloning. Resulting plasmids were pBJB20 (Peft-3::IMMT1 (N-terminal 187 amino acids)::Mac::GFP) and pBJB16 (Pmyo-3::IMMT1 (N-terminal 187 amino acids)::Mac::GFP), and Sanger sequencing was used to confirm plasmid sequences (Eurofins Genomics). Animals were transformed by plasmid DNA microinjection with pha-1 (+) selection in a pha-1 (e2123ts) temperature-sensitive mutant strain, where transgenic animals were selected for growth at 20° C.

C. elegans Strains Growth and Maintenance

All animals were maintained at 20° C. on nematode growth medium (NGM) seeded with OP50 E. coli. Young adult hermaphrodite animals were used for all experiments. Transgenic strains were generated by plasmid DNA microinjection. Where indicated, OP50 was supplemented with all trans-retinal to a final concentration of 100 μM on seeded NGM plates. Animals were cultured on ATR-containing plates for at least one generation. The strain APW138 was generated by homology-directed genome editing through CRISPR/Cas9. Briefly, the endogenous IMMT1 protein was fused with the red fluorescent protein mCherry with a linker region on the C-terminus. mCherry was amplified from plasmid DNA pCFJ90 (forward amplification primer: CTCGCCCAGCTTCTTGTGGCTCACGCCGCCGTCTCATCGATTCGCTCAACTTATCCTC GTAGCCCTCGGACTCCTCGTAGCATGGTCTCAAAGGGTGAAGA (SEQ ID NO:21), reverse amplification primer: TTTTAAAAAACGGTGACGCAAGACAATCAATTGTTCTACTTATACAATTCATCCATG CC (SEQ ID NO:22)) and microinjected into C. elegans hermaphrodite gonads with purified Cas9 protein and crRNA (CTAATAAGTTGAGCGAATCG (SEQ ID NO:23), DNA target) to achieve transgene insertion into the genome of progeny. The transgenic line generated by CRISPR/Cas9 was outcrossed four times to the wild-type strain.

### Fluorescence Microscopy

Images were taken on a FV1000 Olympus laser scanning confocal microscope using a 60× oil objective (Olympus, N.A. 1.42). Diode laser illumination was at 561 nm for red fluorescence and 488 nm for green fluorescence. Where indicated, animals were stained with 10 μM MitoTracker™ Red CMXRos for 4 h. MitoTracker™ stain was dissolved in DMSO, diluted in M9 media (22 mM KH₂PO₄, 42 mM Na₂HPO₄, 86 mM NaCl, 1 mM MgSO₄, pH 7), and added to OP50 seeded NGM plates (DMSO <0.02% final) and allowed to dry. Line scan pixel intensity was performed using ImageJ software. Fluorescent determination of mtON localization was performed according to standard protocols. Briefly, cross-section intensity plots of muscle mitochondrial fluorescence (coexpressing either mtON::GFP and mCherry, or mtON animals stained with MitoTracker™) were smoothed by three-point moving averages and then normalized to maximum intensity of each intensity trace. Distance between maximum red and green signal was calculated, as well as the distance between inflection points (defined as a threshold of 10% increase in pixel intensity from the previous point, in the direction from outer border toward the middle of the mitochondrion).

### Mitochondria Isolation

Caenorhabditis elegans mitochondria were isolated from day 1 adults using differential centrifugation in mannitol and sucrose-based media. Animals from three 15-cm culture plates were transferred into 50 ml of M9 media in a conical tube and allowed to settle by gravity on ice. Pelleted animals were rinsed with ice-cold M9 twice and once with ice-cold mitochondrial isolation media (220 mM mannitol, 70 mM sucrose, 5 mM MOPS, 2 mM EGTA, pH 7.4) with 0.04% BSA. After settling by gravity, supernatant was removed and worms were transferred to an ice-cold mortar containing ˜2 g of pure sea sand per 1 ml of animals. Animals were ground with an ice-cold pestle for 1 min and extracted from the sand using mitochondrial isolation media and transferred to a 10-ml conical tube. The suspension was then transferred to an ice-cold glass Dounce homogenizer and homogenized with 40 strokes. The homogenate was centrifuged at 600 g for 5 min. Supernatant was transferred to a new tube and centrifuged at 700 g for 10 min. The pellet was resuspended in 1 ml of mitochondrial isolation media without BSA, which was centrifuged at 7,000 g for 5 min. The pellet was finally resuspended in 50 μl of mitochondrial isolation media without BSA. Protein concentration was quantified using the Folin-phenol method.

### Light Sources

Illumination sources included a 580 nm Quantum SpectraLife LED Hybrid lamp by Quantum Devices, Barneveld WI, USA (abbreviate) Quantum LED), a 540-600 nm GYX module, XCite LED1 by Excelitas, Waltham MA, USA (abbreviated XCite LED), and a 540-580 nm excitation filter MVX10 Fluorescence MacroZoom dissecting microscope by Olympus (abbreviated MVX). Light intensities are indicated for each experimental condition and were determined with a calibrated thermopile detector (818P-010-12, Newport Corporation, Irvine, CA) and optical power meter (1916-R, Newport Corporation).

### Immunoblotting

Synchronized young adult animals were exposed to 1 Hz light (Quantum LED, 0.02 mW/mm2) for 4 h, and immediately harvested with ice-cold M9 media and centrifuged at 1,000×g for 1 min. Animals were ground by plastic pestle disruption in lysis buffer (20 mM Tris-HCl, 100 mM NaCl, 1 mM EDTA, 1 mM DTT, 10% glycerol, 0.1% SDS, pH 7.6, 1×Halt™ protease inhibitor cocktail, Thermo78429) and diluted 1:1 in sample loading buffer (100 mM Tris-HCl, 10% v/v glycerol, 10% SDS, 0.2% w/v bromophenol blue, 2% v/v b-mercaptoethanol). These samples were heated at 95° C. for 5 min. Isolated mitochondrial samples were prepared as described above and diluted 1:1 in sample loading buffer with 1% SDS. 30 1 g samples were loaded to 7.5% polyacrylamide gels and separated by SDS-PAGE. Proteins were transferred to nitrocellulose membranes and blocked in 5% non-fat milk/TBST (50 mM Tris, 150 mM NaCl, 0.05% Tween 20, pH 8.0) for 1 h at room temperature. Membranes were incubated at 4° C. in primary antibodies diluted 1:1,000 in 5% bovine serum albumin:anti-GFP (ClonTech Living Colours #ab632375), anti-ATP5a (Abcam, #ab14748), 1:60 anti-HSP60 (Department of Biology, Iowa City, IA 52242; Developmental Studies Hybridoma Bank, University of Iowa Department of Biology, Iowa City, IA 52242), (Cell Signaling #4188), anti-Actin (Abcam #ab14128), and 1:10,000 antiphospho-AMPKa Rabbit (Cell Signaling, #2535). Membranes were washed in TBST and incubated in horseradish peroxidase-conjugated secondary antibodies: 1:2,000 anti-rabbit IgG (Cell Signaling #7074S) or anti-mouse IgG (Thermo Scientific #32430, lot #RF234708) for 1 h at room temperature. Proteins were visualized using ECL (Clarity Western ECL Substrate, Bio-Rad) by chemiluminescence (ChemiDoc, Bio-Rad). Densitometry was performed using Image Lab software (version 5.2.1).

### Protease Protection Assay

Inner membrane localization was determined. Briefly, mitochondria were isolated as described and pelleted at 7,000 g for 5 min, and then, the supernatant was removed. Mitochondria were resuspended in hypotonic swelling buffer (20 mM HEPES, 1 mM EGTA, pH 7.2 at 4° C.) and incubated on ice for 10 min. Mitochondria were then pelleted again at 7,000 g for 5 min and then resuspended in MRB. Samples were treated with proteinase K (No. P81075; Biolabs) at 0.08 U/mg protein for 10 min with or without Triton X (2.5% v/v), and then, digestion was inhibited with the serine protease inhibitor, phenylmethylsulfonyl fluoride (PMSF, 40 mM). Sample loading buffer was added 1:1, and the SDS-denatured protein lysates were resolved on a 7.5% polyacrylamide gel as described above.

### Mitochondrial Membrane Potential Measurement

Isolated mitochondria at 0.5 mg/ml were stirred in mitochondrial respiration buffer (MRB: 120 mM KCl, 25 mM sucrose, 5 mM MgCl2, 5 mM KH₂PO₄, 1 mM EGTA, 10 mM HEPES, 1 mg/ml FF-BSA, pH 7.35) at 25° C. in the presence of 2 μM rotenone and 5 mM succinate where indicated. 300 nM tetramethylrhodamine ethyl ester (TMRE, Thermo Fisher, T669) was added to observe mitochondrial membrane potential in quench mode. Under quenching conditions, TMRE fluorescence is low in the presence of a AWm. Upon addition of a protonophore (e.g., FCCP), TMRE will exit mitochondria and dequench and increase total fluorescence. TMRE signal was measured by Cary Eclipse Fluorescence Spectrophotometer (Agilent Technologies) using a 335-620 nm excitation filter and a 550-1,100 nm emission. Illumination was performed continuously throughout all measurements (555 nm, 0.0016 mW/mm²). Increasing illumination time exposed mitochondria to more photons (calculated as fluence, J/cm²). After stable baseline measurements with or without succinate, 2 M FCCP was added to completely depolarize mitochondria. The average fluorescence intensity after addition of 2 μM FCCP (maximum fluorescence in quench mode) was subtracted from the starting test condition to give a change in fluorescence corresponding to changes in ΔΨₘ (ΔF for conditions without succinate, and ΔFₘₐₓ for conditions with succinate). To represent polarization of the ΔWₘ, the ratio of change in fluorescence (DF, FCCP fluorescence minus experimental fluorescence) was used to the maximum change in fluorescence generated by succinate-driven Δψₘ (ΔFₘₐₓ).

### Mitochondrial Matrix pH Measurement

The ratiometric pH indicator BCECF-AM (Thermo Fisher, B1170) was used to measure pH changes in the mitochondrial matrix in response to succinate respiration or mtON activation. Isolated mitochondria (˜200 μl per isolation) were incubated at room temperature with 50 μM BCECF-AM for 10 min with periodic mixing. Mitochondria were then pelleted at 7,000 g for 5 min at 4° C., and isolation media replaced and pelleted again to remove extramitochondrial BCECF-AM. Isolated mitochondrial suspensions were then assayed under the same conditions as in the mitochondrial membrane potential measurements described above. Ratiometric fluorescent signal was measured by Cary Eclipse Fluorescence Spectrophotometer (Agilent Technologies) using 440 and 490 nm excitation wavelengths and 545 nm emission. The fluorescence intensity ratio at 545 nm of 490/440 nm excitation wavelengths was used to represent pH changes in the mitochondrial matrix. Light treatment was 0.16 J/cm² (XCite LED, 0.02 mW/mm²), and 2 μM FCCP was used at the end of each trace to establish baseline signal.

### ATP Measurement

Relative ATP levels were determined in isolated mitochondria given a known amount of ADP to test the ability of mtON to drive ATP synthesis. A luciferase bioluminescence kit was used according to the manufacturer's instructions (Invitrogen™ Molecular Probes™, A22066). Mitochondria were stirred in MRB at 0.5 mg/ml with 1 mg/ml fat-free BSA, 600 μM ADP, and 2 μM rotenone. 5 mM succinate was used for a control for maximum ATP level, and 0.001 mg/ml oligomycin A was used as a zero ATP synthesis control. Mitochondrial suspensions were immediately frozen with liquid nitrogen after 1, 5, or 10 min light exposure (XCite LED, 0.02 mW/mm²). Samples were then thawed on ice, centrifuged at 14,800 g, and supernatant was collected and run at 1:100 dilution in MRB in the luminescence assay. Oligomycin A control values were subtracted from experimental reads, and data were then normalized to luminescent signal from succinate control samples (complete ADP conversion confirmed by monitoring 02 consumption rate transitions).

### Mitochondrial O₂ Consumption

O₂ consumption was measured using a Clark-type O₂ electrode (S1 electrode disk, DW2/2 electrode chamber and Oxy-Lab control unit, Hansatech Instruments, Norfolk UK) at 25° C. Isolated mitochondria were stirred in MRB at 1 mg/ml with 1 mg/ml fat-free BSA. Substrates and inhibitors were added by syringe port (100 μM ADP, 2 μM rotenone, 5 mM succinate). Given excess succinate as substrate for ETC respiration, we measured the amount of O₂ required to convert 50 nmol of ADP to ATP and established a baseline for comparison. To test mtON activity, mitochondria were illuminated as in the ATP measurement, for 1, 5, or 10 min in the presence of ADP without succinate to allow for mtON conversion of ADP to ATP. Any remaining ADP was then converted using 02-dependent ETC respiration upon succinate addition. Slopes were calculated from plots of O₂ concentration versus time to give rates of 02 consumption during ADP respiration, ADP+succinate respiration, and respiration after ADP had been entirely consumed. The intersections of these three rates were used to calculate total amount of O₂ consumed during ADP+succinate respiration. Light activation of mitochondria (XCite LED, 0.02 mW/mm²) during ADP respiration alone was carried out for differing lengths of time to test the ability of mtON to drive ADP consumption before succinate was added. Dark control was 10 min of no illumination before addition of succinate.

### ETC Inhibitor Assays

Experiments were performed on at least three separate days, using 15-100 young adult animals per plate. Seeded plates were supplemented with rotenone (50 μM final concentration), antimycin A (50 μM final concentration), or oligomycin A (31 μM final concentration) 24 h before animals were transferred onto them. For azide toxicity, animals were placed in M9 buffer with 250 mM azide. Control plates were kept in the dark, and experimental plates were exposed to 1 Hz light (Quantum LED, 0.02 mW/mm²) for the duration of the experiment. For all toxins, animals that were moving or those that moved in response to a light touch to the head were scored as alive. For rotenone, surviving animals were scored after 5 h. For antimycin A, animals were scored 16 h after exposure. For azide, animals were exposed for 1 h in M9 and allowed to recover for 1 h on a seeded culture plate and then survival was scored. Azide experimental plates were exposed to light (XCite LED, 0.19 mW/mm²) for the duration of azide treatment and recovery. For oligomycin, animals were scored 18 h after exposure. For oligomycin A, animals were scored 18 h after exposure.

### Locomotion Assay

Locomotion was scored by counting the number of body bends in 15 s immediately after being transferred off OP50 food (n=36-46 animals scored on at least 2 separate days). One body bend was scored as a deflection of direction of motion of the posterior pharyngeal bulb. For AMPK activation, animals were placed on plates containing 1 mM AICAR 4 h before counting body bends. AICAR was dissolved in M9 buffer, added directly onto OP50 seeded plates, and allowed to dry. Illumination was continuous through measurements (MVX, 0.265 mW/mm²).

### Mitochondrial DNA PCR

The uaDf5 mitochondrial DNA deletion was detected by PCR amplification as described from whole-animal lysate. Briefly, using primers for wild-type mitochondrial DNA (forward: TTGGTGTTACAGGGGCAACA (SEQ ID NO:24), reverse: CTTCTACAGTGCATTGACCTAGTC (SEQ ID NO:25), expected size: ˜500 bp) and for uaDf5 DNA (forward: CCATCCGTGCTAGAAGACAA (SEQ ID NO:26), reverse: CTTCTACAGTGCATTGACCTAGTC (SEQ ID NO:27), expected size ˜299 bp), PCR was performed as follows: 98° C. for 30 s for melting, 55° C. for 30 s for annealing, and 72° C. for 30 s for elongation, for 35 cycles. Mitochondrial DNA was visualized by electrophoresis through 2% agarose gel stained with ethidium bromide.

uaDf5 Progeny Survival

Single L4 animals were placed on seeded plates and allowed to develop through egg laying. The number of progeny was scored after 4 days, where light treatment was throughout (Quantum LED, 0.02 mW/mm²) for the duration of the experiment for +light conditions. Progeny were scored as the number of animals that developed to L4 stage.

### Hypoxia and Reoxygenation

Experiments were carried out using a hypoxic chamber (Coy Laboratory Products, 5%/95% H₂/N₂ gas, palladium catalyst) at 26° C. with 50-100 animals per plate. O2 was <0.01%. Hypoxic preconditioning (PC) duration was 4 h, and control animals were incubated at 26° C. in room air for the same time. 1 Hz illumination (Quantum LED, 0.02 mW/mm²) was carried out during PC period or during the hypoxic period. Hypoxic exposure was for 18.5, 21 h after PC. Twenty-four hours after hypoxia exposure, animals that were moving or those that moved in response to a light touch to the head were scored as alive. Data from days where PC was at least 15% effective for both +ATR and −ATR were used. Animals supplemented with ATR were allowed to lay eggs onto plates with no ATR that were subsequently used for HR experiments to minimize confounding effects of ATR. ATR from the parent animal was sufficient to provide active mtON in progeny as tested by the azide toxin assay described above. Only progeny from parents supplemented with ATR were significantly protected against azide upon illumination (tested by one-way ANOVA, −ATR, light [17.3% survival] versus+ATR, +light [66.8% survival] P=0.0054, ATR, +light [32.5% survival] versus +ATR, +light P=0.0395, +ATR, −light [27.5% survival] versus+ATR, +light P=0.0199, DF=11, F=8.94). PC experiments were represented as protection (%), where baseline survival was subtracted to correct for protection from ATR (−ATR survival: 34.4+/−14.4%, +ATR survival: 49.7+/−20.3%, 2-sample 2-tailed paired t-test, P=0.033, n=12 independent experiments). FCCP dissolved in ethanol was deposited onto seeded NGM plates.

### Statistics

One-way ANOVA was used when comparing only the four experimental conditions. Two-way ANOVA was used when comparing the conditions with other variables, such as +/−succinate. Shapiro-Wilk normality tests were used to determine whether parametric or non-parametric tests should be used.

### Example 2: Light-Activated Proton Pump Mitochondria-ON (mtON) is Expressed in Mitochondria

Expression of mtON was directed to the mitochondrial inner membrane in C. elegans using a fusion of the proton pump to a mitochondrial targeting sequence of the IMMT1 protein (FIG. 2). Using a C-terminal mtON::GFP fusion for subcellular visualization, overlap was observed of green and red fluorescence in C. elegans tissues stained with MitoTracker™ CMXRos. The expression of mtON was confirmed in isolated mitochondrial preparations by immunoblot against GFP and observed a band at the predicted molecular weight of 82 kDa. mtON activation increases the PMF.

The proton pump Mac can generate proton gradients in response to light. Likewise, mtON in isolated mitochondria caused a light dose-dependent energized PMF in response to 550-590 nm light that matched the PMF generated by the ETC. Increased PMF was observed through both ΔΨm and ΔpH components. These results indicated a PMF was generated using only the energy from light, bypassing the ETC, as no source of electrons (metabolic substrates to generate electron donors for respiratory function) was required. To further test this, oxygen consumption was measured in isolated mitochondria fueled with substrates to drive respiration. When activated, mtON decreased reliance on oxygen to make ATP, demonstrating that mtON can supplement ETC activity by using light rather than oxygen and electrons. This showed that this tool can be used to control the PMF independent of oxygen or substrate availability.

### Example 3: MtON Alters Mitochondrial Function and can Restores Lost Mitochondrial Function

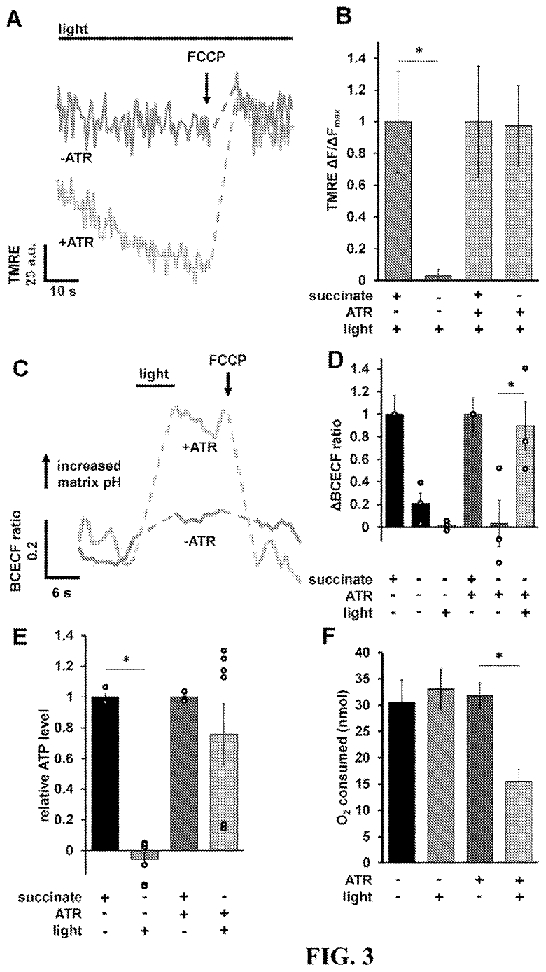

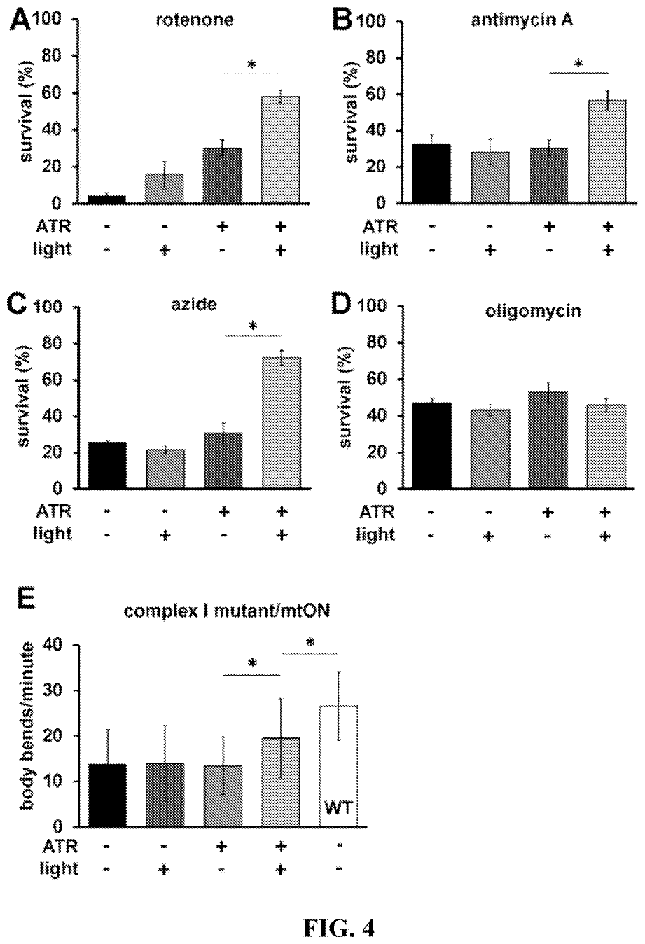

Proton pumping activity of mtON requires the cofactor all trans-retinal (ATR). Because C. elegans do not produce ATR endogenously, exogenous supplementation is required for the light-activated proton pump to function. The expression of mtON (and the included GFP) was controlled for in functional and non-functional forms, depending on whether ATR was supplemented, in addition to the use of light controls. It was demonstrated that mtON could generate a PMF by measuring the Δψm component using the indicator tetramethylrhodamine ethyl ester (TMRE) in isolated C. elegans mitochondria (FIG. 3, panel A and panel B). Under non-phosphorylating conditions with the ETC inhibited with rotenone and in the absence of added substrate to fuel ETC activity, mtON activation was able to polarize the Δψm as much as ETC-driven respiration light dose dependently. Light activation of the non-functional pump (no ATR) had no effect on Δψm (FIG. 3, panel B). pH changes were measured in the mitochondrial matrix in response to light as another readout of mtON activity. Using a ratiometric pH indicator, BCECF-AM, matrix pH increased in response to mtON activation, mimicking the effect of succinate driven respiration (FIG. 3, panel C and panel D). Mitochondria generate ATP via respiration and it was demonstrated that mtON increases ATP synthesis without respiration. ATP levels were measured from isolated mitochondria that were supplied with ADP to phosphorylate. As expected, mtON activation increased ATP levels (FIG. 3, panel E and panel F) light-dose dependently.

Given that mtON activity decreased reliance on the ETC in vitro, it was tested if mtON could compensate for acute ETC dysfunction in vivo. C. elegans were exposed to toxic inhibitors of specific sites within ETC complexes and scored their survival in response to mtON activation. The data (FIG. 4) indicated that mtON can partially overcome inhibited ETC activity in whole organisms, but not direct inhibition of ATP synthesis downstream of the PMF, as expected. To further test if mtON could compensate for a dysfunctional ETC, it was expressed in a complex I mutant background (gas-1). mtON activation was able to partially rescue locomotion in the gas-1 mutant background, suggesting the impaired locomotion is due to the reported decreased PMF.

### Example 4: MtON Alters In Vivo Energy Signaling Pathways

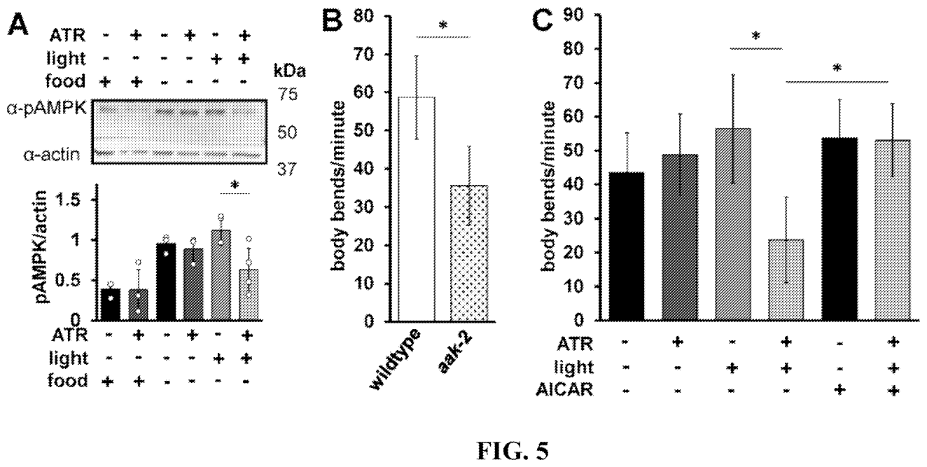

It was tested whether mtON could affect metabolic signaling. One way organisms sense energy availability and preserve energy homeostasis is through the AMP-activated protein kinase (AMPK). In C. elegans the aak-2 gene encodes the catalytic subunit ortholog of mammalian AMPKα2. Mutation of aak-2 has well-characterized phenotypic outputs linked to energy availability, and serves as a regulator of whole-organism energy sensing. It was hypothesized that mtON activity would signal energy availability and decrease phosphorylation of AMPK, its activated state. As such, animals expressing mtON were exposed to light in the absence of food, where AMPK should be phosphorylated, and immunoblotted against phosphorylated AMPK. It was found that removal from food increases AMPK phosphorylation as expected, and mtON activation prevented this phosphorylation (FIG. 5). In C. elegans, the AMPK homologue AAK-2 regulates a behavioral response to food availability, where in the absence of food animals will increase locomotion to search for food. It was found that mtON activation attenuated the energy deficit signal for animals off food, suppressing their movement, and thus mimicking the aak-2 loss-of-function phenotype. These data suggest that mtON activity can modify downstream metabolic signaling.

In addition to supplementing ETC activity, mtON was able to compensate for lost ETC activity. In a whole-animal C. elegans model, animals exposed to ETC toxins survived more when mtON was active compared to controls, indicating mtON activation can overcome dysfunctional respiration. These results were confirmed using a mitochondrial mutant strain with decreased animal locomotion, a phenotype rescued by mtON activation. Impaired energetic function of mitochondria is signaled throughout cells and organisms, and AMP-activated protein kinase (AMPK) is a molecular energy sensor responsible for some of this signaling. mtON silenced AMPK activation under starvation conditions and decreased animal locomotion when starved. These acute changes in AMPK signaling could have broad and lasting impacts on metabolism and physiology.

### Example 5: MtON Inhibits Hypoxia-Adaptation Function

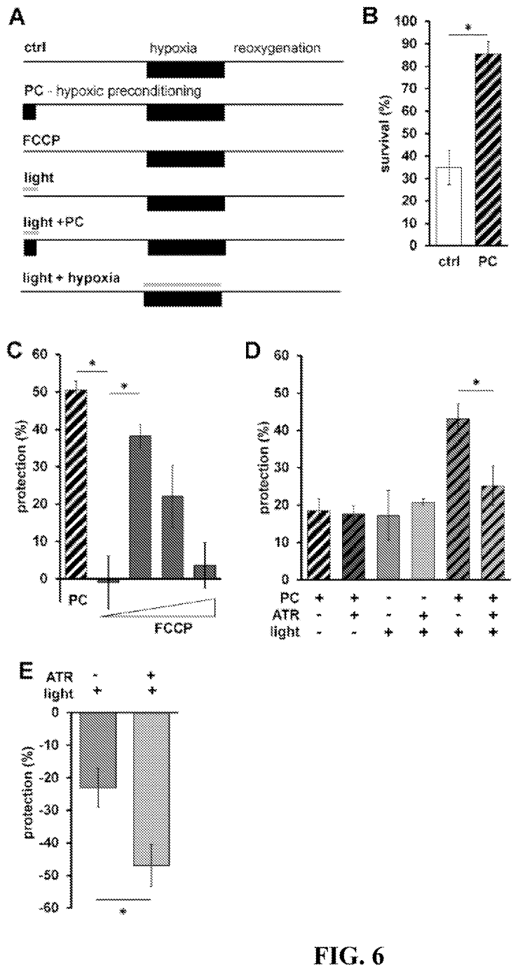

Hypoxia and reoxygenation (HR) is a pathologic insult that involves changes in the PMF that can contribute to injury and survival depending on the context and degree of (de) polarization. The phenomenon of preconditioning (PC) is effectively modeled with hypoxia in C. elegans, where a short period of hypoxia protects against a later pathologic exposure (FIG. 6) and is thought involve changes in the PMF. mtON was used to demonstrate the sufficiency of PMF loss to protect against hypoxia (FIG. 6). These findings that protection afforded by PC relies on the transient loss of PMF suggest that a decreased PMF is sufficient to elicit stress resistance at a later period. This implies that interventions at the level of the PMF alone can impact cellular responses to stress. Combining the mtON approach with tissue-specific gene promoters, precise spatiotemporal control could be achieved in discerning the effects of mitochondrial function across tissues in whole organisms.

One example of a lasting impact downstream of AMPK and other signaling pathways is the ability to adapt to stress. mtON was used to study hypoxia adaptation as a readout of stress resistance. In C. elegans and in mammals, a short exposure to hypoxia is protective against a later, more damaging exposure. The protection conferred therein can be suppressed by mtON activation, suggesting that a decreased PMF during the short hypoxia is required for its protective effect. PMF dissipation triggers stress resistance prophylactically. The mtON system highlights how temporal control of the PMF can reveal fundamental requirements for stress resistance in models of mitochondrial and metabolic dysfunction.

### Example 6: Extending Healthy Lifespan and the Amelioration of Age-Associated Damage Through the Conversion of External Light Energy into ATP Synthesis

Decline of Mitochondrial Function with Age

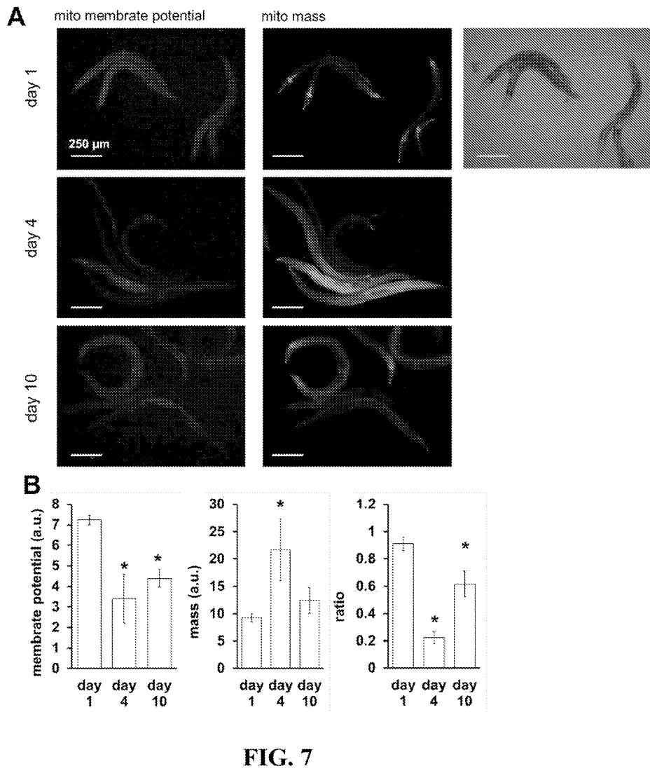

Increasingly, biological age is recognized as decline in cellular functions that can be reversed. For example, mitochondrial function and PMF declines with age (FIG. 7, panel A and panel B). This decline is shown here and documented in many model organisms, and is therefore thought to be an early indicator of functional decline with age. However, this process can be viewed as biphasic, where early aging state is in fact coupled with increased mitochondrial activity (e.g. oxygen consumption rate), which generates mitochondrial stress that eventually leads to age-associated mitochondrial decline.

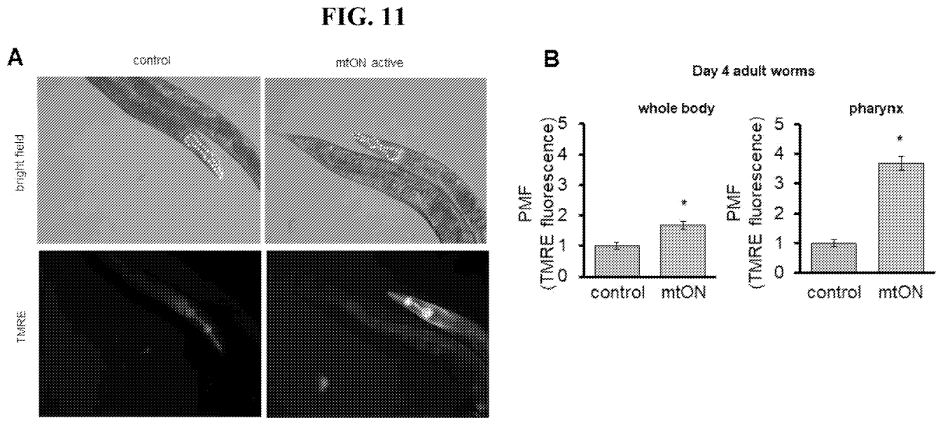

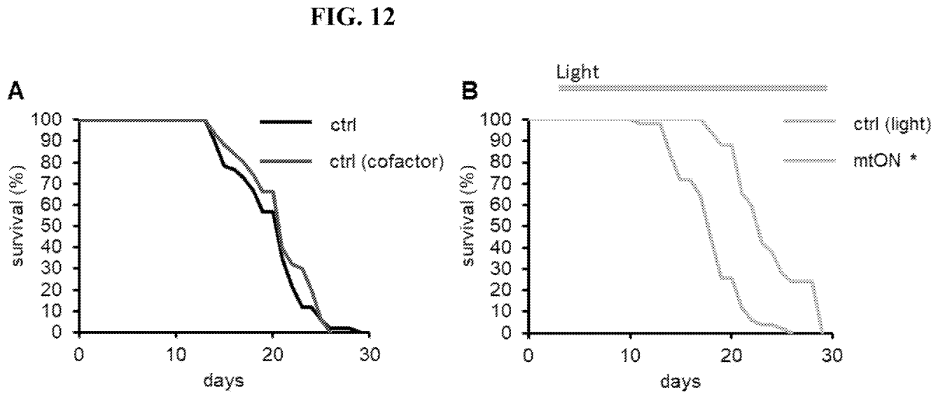

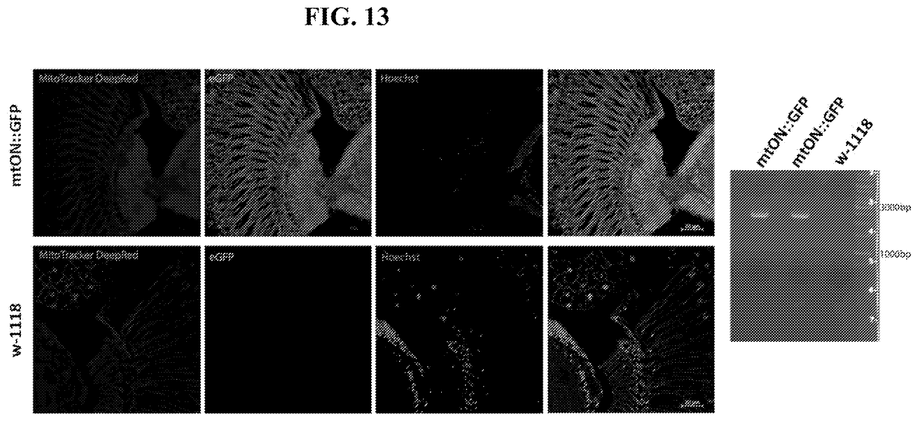

A transgenic C. elegans strain that has one copy of the mtON transgene using CRISPR/Cas9 was generated. FIG. 11 demonstrates that mtON activation can restore the mitochondrial function in aged worms, as assessed by the protonmotive force. FIG. 12 demonstrates that activation of mtON can extend lifespan.

Drosophila melanogaster flies were generated capable of expressing mtON and confirmed that the mtON signal colocalizes with mitochondria. mtON flies, supplemented by ATR and light have extended life span and attenuated onset of hallmarks of aging.

Restoration of Membrane Potential and Increase in Lifespan Using mtON

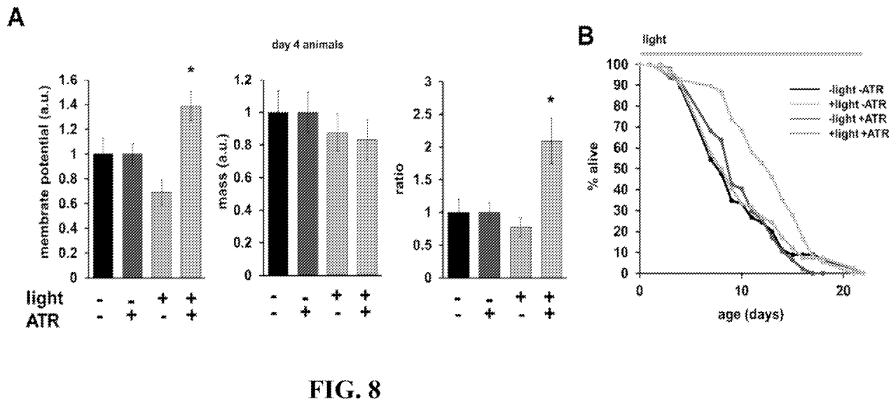

A test sought to reverse the loss of PMF at day 4 of adulthood when the most severe loss of PMF is observable. Using mtON activation for 4 days into adulthood, it was found that whole-animal measures of PMF were significantly increased compared to age-matched control populations (FIG. 8, panel A). Further, mtON activation throughout life caused a significant increase in lifespan compared to control populations (FIG. 8, panel B). These results show that using optogenetic PMF rescue is a feasible intervention to reverse the age-related loss of PMF and cause increased lifespan.

### Example 7: Optical Control of T Cell Metabolism

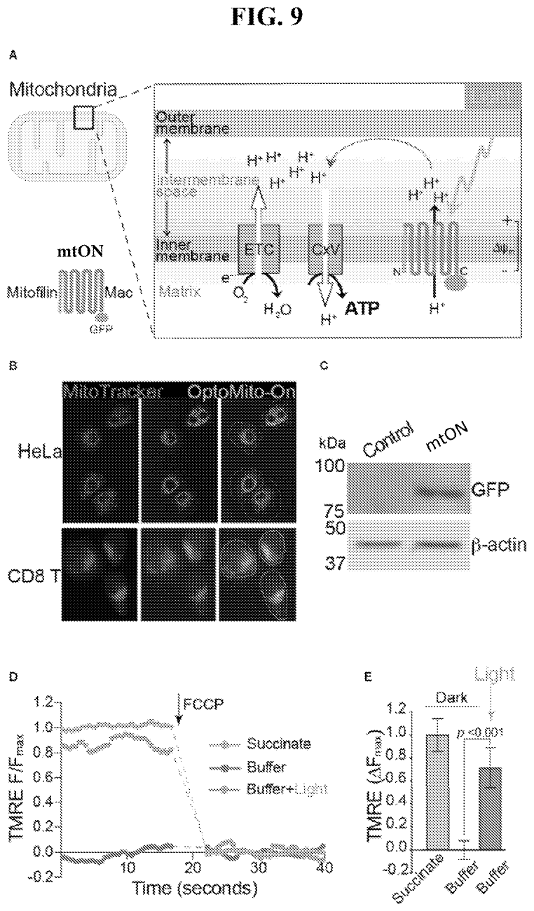

Using mtON in mouse T cells, it was shown that it is possible to remotely control T cell metabolism and effector functions (see e.g., Amitrano A M et al., Front Immunol. 2021 12:666231. doi: 10.3389/fimmu.2021.666231. PMID: 34149701, which is incorporated herein by reference). A genetically encoded photoactivatable proton pump that is expressed in the inner mitochondrial membrane (FIG. 9, panel A). The mtON construct consists of the light-driven proton pump, ‘Mac,’ that was derived from the fungus Leptosphaeria maculans and is fused to the N-terminal mitochondrial targeting domain of the mitochondrial inner membrane protein Mitofilin, which delivers and orients Mac in the membrane (FIG. 9, panel A). During OxPhos, electrons from NADH and FADH2, products of cellular metabolism, enter the mitochondrial electron transport chain (ETC), located in the inner mitochondrial membrane. As electrons are transferred through the ETC to oxygen, protons are pumped from the mitochondrial matrix to the intermembrane space, thereby generating a protonmotive force that is composed of an electrical (Δψm) and chemical (ΔpH) gradient across the inner membrane (FIG. 9, panel A).

### Expression and Functions

HeLa cells were transfected with the mtON-GFP construct and labeled cells with MitoTracker (red) to specifically stain the mitochondria. Confocal microscopy confirmed that there was a high degree of overlap between GFP signals (mtON) and red fluorescent signals (mitochondria), indicating that the mtON protein is successfully expressed in the mitochondria (see FIG. 9, panel B). Expression of the full-length mtON construct was further confirmed by Western blot analysis of cell lysates prepared from HEK293T cells that expressed mtON (82 kDa) (FIG. 9, panel C).

Stimulation of mtON with light results in polarization of the mitochondrial inner membrane. To determine whether mtON reaches the inner mitochondrial membrane in the correct orientation and to confirm the functional responsiveness of mtON, the study measured the mitochondrial membrane potential (Dym) using TMRE. The study first isolated mitochondria from HEK293T cells expressing mtON and treated them with succinate, a complex II ETC substrate, to generate a maximal mitochondrial membrane potential as a positive control (FIG. 9, panel D). In the absence of succinate (a negative control), the mitochondria did not generate a membrane potential. However, stimulation with 540-600 nm light was sufficient to rescue the mitochondrial membrane potential in the absence of succinate, indicating the expression and correct orientation of functional mtON in mitochondria (FIG. 9, panel D, panel E).

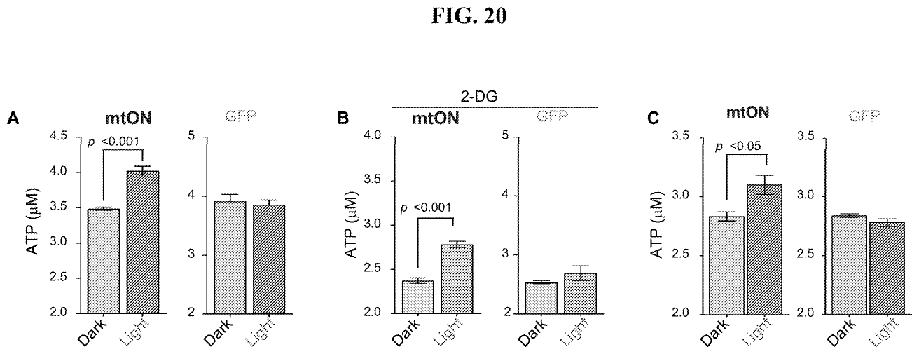

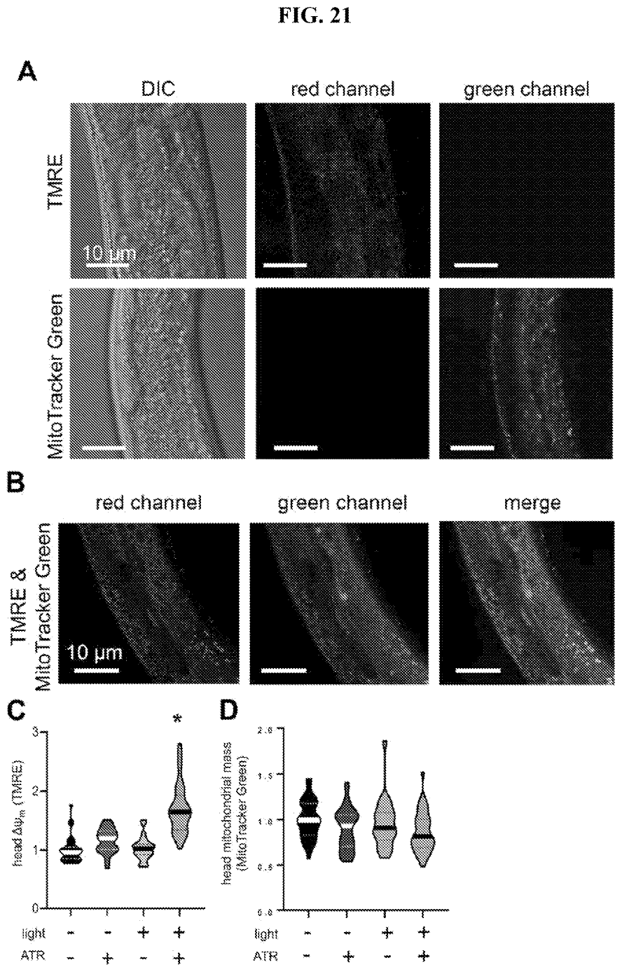

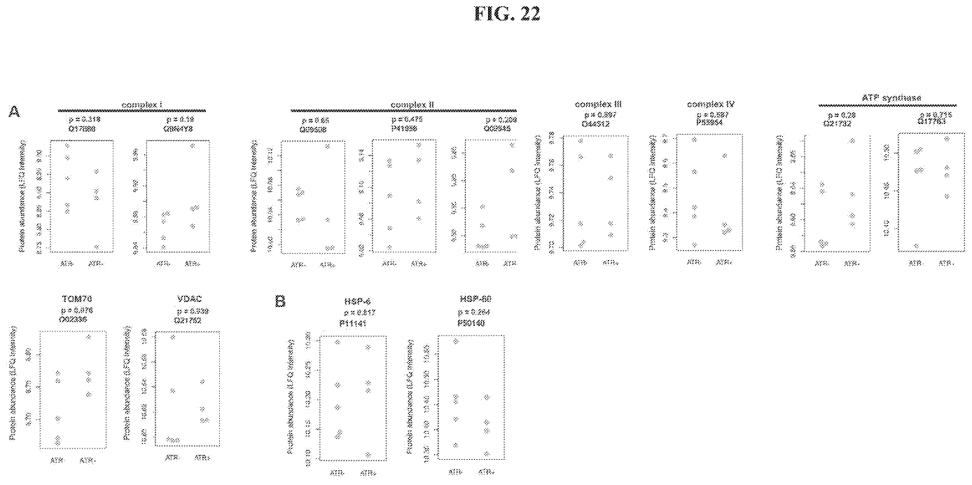

To test whether mitochondrial polarization driven by light-activated proton transport leads to enhanced ATP production (FIG. 9, panel A), the study transfected HEK293T cells and mouse CD8+ T cells with mtON, illuminated the cells (590±10 nm, 0.32 mW/mm² at 1 Hz), and immediately lysed them to detect ATP. 2-Deoxy-D-glucose (2-DG), a glucose analog, was used to inhibit glycolysis and thus mimic the glucose-deficient TME. Optical stimulation of mtON expressing HEK293T cells with or without 2-DG treatment yielded a significant increase in ATP compared with that of cells cultured in the dark (FIGS. 20, panels A, B). Stimulation of GFP-expressing cells with light did not alter ATP levels, indicating that stimulation with light at 590 nm was not detrimental to cell viability. The study observed a similar outcome in mtON expressing CD8+ T cells (FIG. 4C).

### Isolated Mitochondria

Based on this model, light stimulation of mtON results in the polarization of the mitochondrial inner membrane. To determine whether mtON reaches the inner mitochondrial membrane in the correct orientation and to confirm the functional responsiveness of mtON, the mitochondrial inner membrane potential (Δψm) was measured using tetramethylrhodamine, ethyl ester (TMRE). TMRE is a lipophilic cationic dye that accumulates in the mitochondrial matrix proportional to the mitochondrial membrane potential. First, mitochondria were isolated from HEK293T cells expressing mtON and treated them with succinate, a substrate for the ETC (complex II), to induce a maximal mitochondrial membrane potential as a positive control. In the absence of succinate (a negative control), the mitochondria did not generate a membrane potential. However, stimulation with yellow light (590±10 nm, 0.32 mW/mm² at 1 Hz) was sufficient to rescue the mitochondrial membrane potential in the absence of succinate, indicating the expression of functional mtON in mitochondria.

### HEK293T and Mouse T Cells

To test whether the mitochondrial polarization driven by light-activated proton transport leads to enhanced ATP production, we transfected HEK293T cells and mouse CD8+T cells with mtON, illuminated (590±10 nm, 0.32 mW/mm² at 1 Hz) them, and immediately lysed to detect ATP. 2-Deoxy-D-glucose (2-DG), a glucose analog, was used to inhibit glycolysis and thus mimic the glucose-deficient tumor microenvironment. Optical stimulation of mtON-expressing cells with or without 2-DG treatment yielded a significant increase in ATP compared with that of the cells in the dark (no light) condition. Light stimulation of GFP-expressing cells did not alter ATP levels, indicating that 590 nm light was not detrimental to cell metabolism. In combination with isolated mitochondria data, these results demonstrate that mtON can be expressed in cells and to permit the photoactivatable control of the mitochondrial membrane potential resulting ATP production.

### T Cell Migration

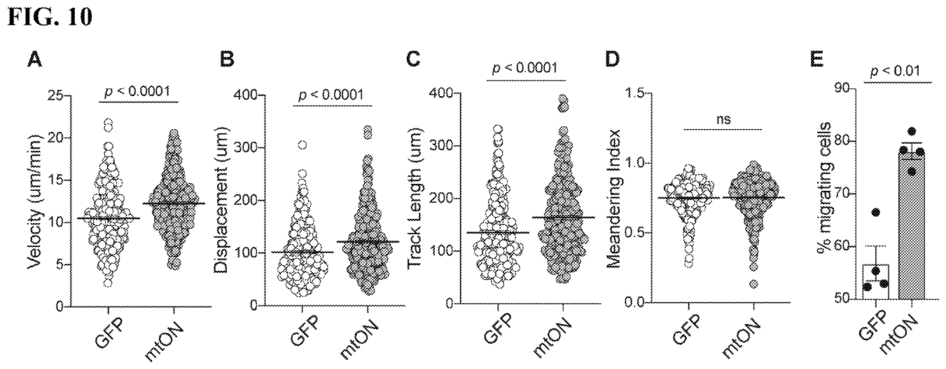

After confirming mtON is expressed correctly, there followed additional functional experiments, specifically looking at cell migration in CD8+ T cells expressing mtON (PA-OxPhos). The migration conditions were the same as in previously described experiments, except we now used the Texas Red filter (˜500 nm) on an epifluorescent microscope to activate mtON while CD8+ T cells are migrating on ICAM-1+CXCL12. CD8+ T cells were sorted based on GFP expression and the mock negative control cells were GFP negative. Both mock and mtON expressing CD8+ T cells were treated identically. The cell tracks were analyzed with Volocity and it was found that there is an increase in the cell velocity as well as the cell displacement and the meandering index in CD8+ T cells expressing mtON (FIG. 10, panels A-D). Additionally, there was a large increase in the overall percentage of migrating CD8+ T cells expressing mtON compared to those expressing GFP (FIG. 10, panel E). This data demonstrates that increasing mitochondrial ATP production with mtON is able to boost CD8+ T cell migration, which can be beneficial in the tumor-killing T cells.

### Example 8: Mitochondria-ON Increased Δψm In Vivo

The voltage potential across the inner membrane of mitochondria (membrane potential Δψm) decreases with age in many model systems. Δψm is a fundamental driver of diverse mitochondrial functions, including adenosine triphosphate (ATP) production, immune signaling and genetic and epigenetic regulation. Decreased Δψm is an attractive explanation for the complex dysfunctions of aging; however, it is unclear whether decreased Δψm is a cause or a consequence of cellular aging.

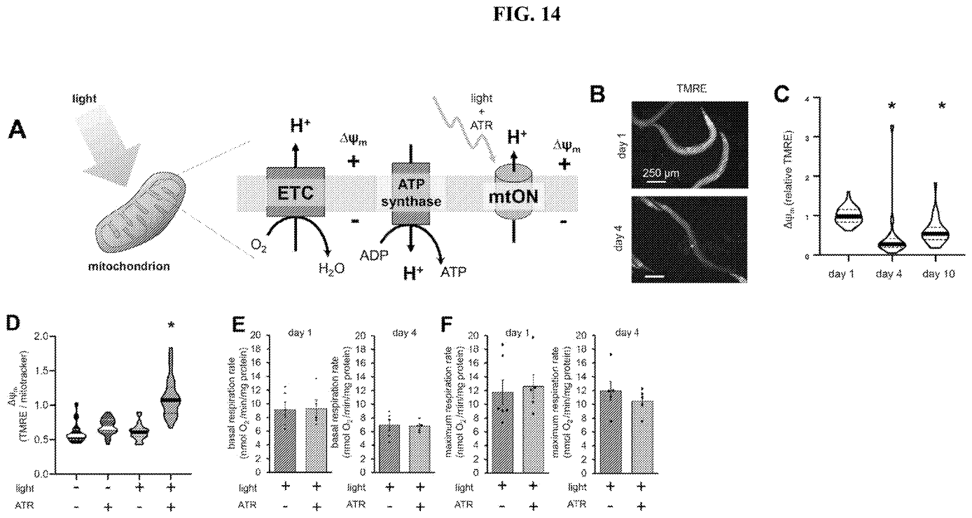

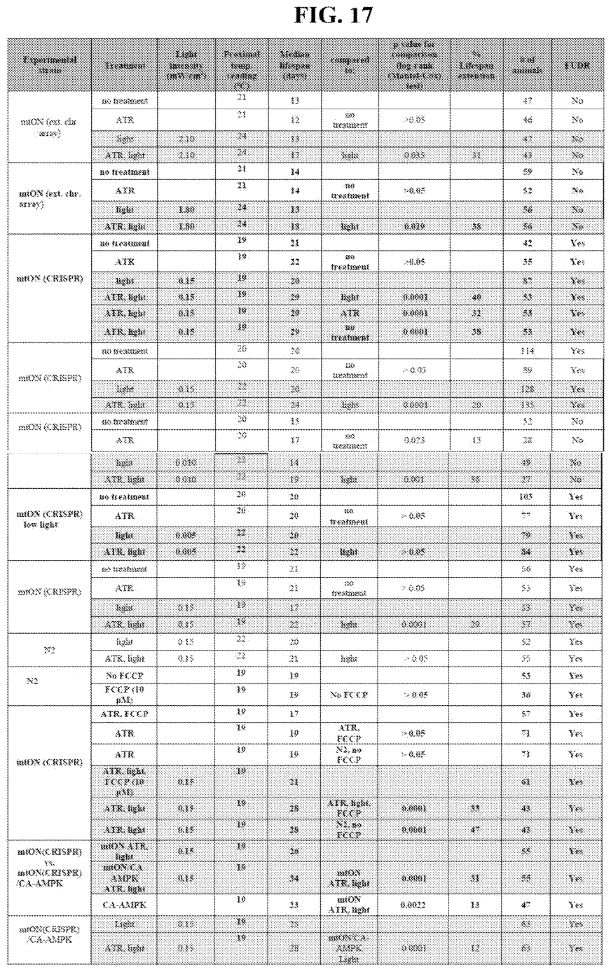

mtON (FIG. 14a) isolates Δψm as a single experimental variable in vivo and requires both light activation and a cofactor, all-trans-retinal (ATR), for proton pumping activity. Caenorhabditis elegans does not produce ATR, allowing for control conditions of light exposure alone (which can be damaging), mtON protein expression alone and ATR supplementation alone (which does not affect lifespan or physiology in this context; FIG. 17). Only animals supplemented with ATR and illuminated will have mtON activity and increased Δψm.

Δψm naturally declines with age in C. elegans (FIG. 14b,c), as expected. mtON activation reversed that decline in two different genetic backgrounds (FIG. 14d and FIG. 11). mtON activation did not impact mitochondrial mass observed by mitochondrial staining and quantitative proteomics. Respiration rates were similar across conditions (FIG. 14e,f) as expected, given mtON's specificity for Δψm alone.

### Example 9: MtON Extended Lifespan and Healthspan

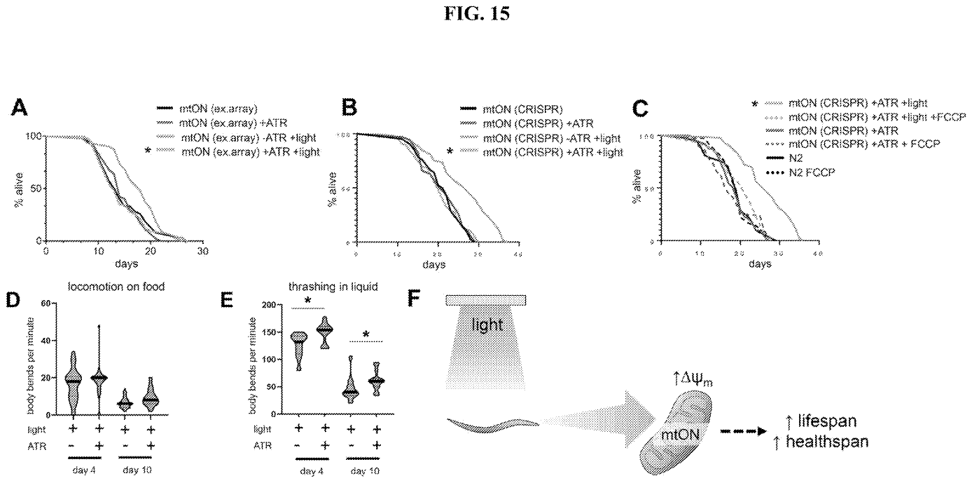

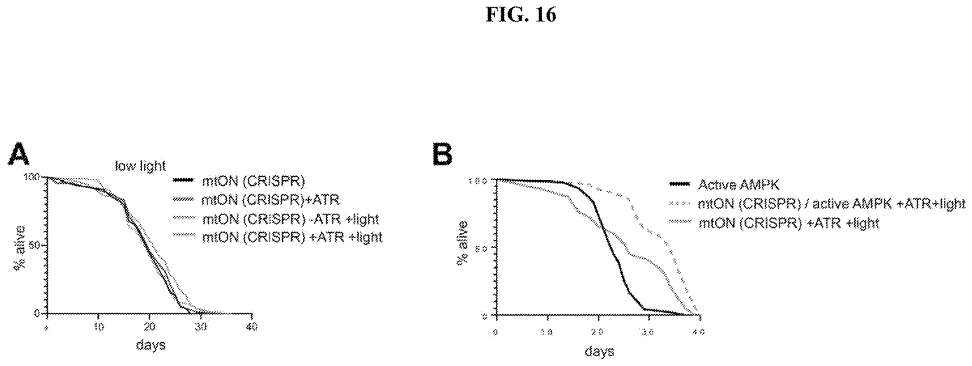

The study activated mtON throughout lifespan beginning in adulthood and found an increase in lifespan compared to controls (FIG. 15a,b). Lifespan extension was replicated independently across three different strains, different light intensities and in different laboratories (FIG. 17) and was sensitive to the mitochondrial uncoupler, carbonyl cyanide p-(trifluoromethoxy)phenylhydrazone (FCCP) (FIG. 15c), which dissipates Δψm. FCCP had no effect on lifespan on its own (FIG. 15c) and ATR and light did not affect lifespan in wild-type animals (FIG. 17). When exposed to light below the threshold to maximally activate mtON9 lifespan was not extended (FIG. 16a). These data together indicate that increasing Δψm causes increased lifespan.

Mild inhibition of mitochondrial function during development (but not during adulthood) extends C. elegans lifespan; conversely, here, we show that attenuating the age-associated decrease in Δψm in adults can extend lifespan. Accordingly, targets of the mitochondrial unfolded protein response did not change after mtON activation. These differences may reflect a lifespan-extending hormetic response from mitochondrial perturbation during development versus the beneficial effects from directly sustaining mitochondrial function during adulthood.

Organisms, including humans and C. elegans, have trouble moving as they age due to physiological decline. This functional decline was mitigated by mtON activation in worms thrashing in liquid, but not on solid media (FIG. 15d,e). These results show that age-associated physiological dysfunction can be improved by reversing the loss of Δψm that occurs with age. How improving Δψm may influence redox metabolites, including NAD+/NADH, which are known to impact biological aging, should be further assessed. To probe a potential signaling pathway, the study tested the effect of mtON in long-lived worms with constitutively active AMPK signaling. mtON further increased lifespan in this model (FIG. 16b and FIG. 17), indicating that increased Δψm can additionally contribute to longevity in parallel with a canonical signaling pathway.

In summary, the study used a technology that harnesses light energy to generate Δψm to test the hypothesis that Δψm causally determines longevity in C. elegans (FIG. 15f). The study showed that preserved Δψm during adulthood is sufficient to slow normative aging and improve at least some functional measures of health.

### Example 10: MtON Lifespan Extension in Invertebrate Drosophila melanogaster

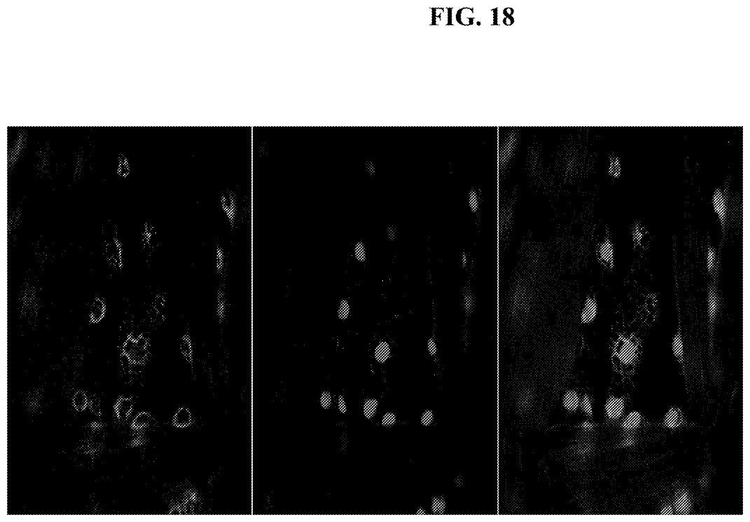

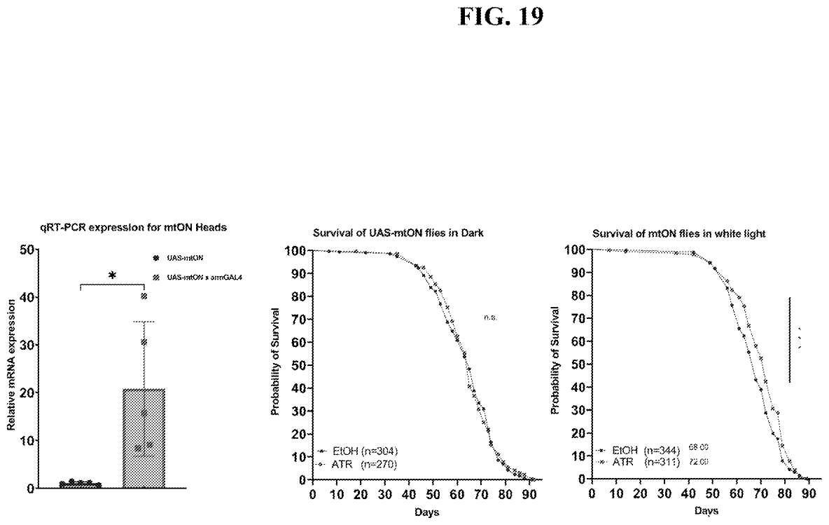

The study provided additional data showing the mitochondrial localization of mtON in Drosophila and that activation of mtON extends Drosophila lifespan (FIGS. 18-19). mtON is expressed in Drosophila melanogaster and colocalizes with a mitochondrial marker. In FIG. 18, the study shows that mtON::GFP is detected in the heads of mtON drosophila and that the GFP signal (eGFP) co-localizes with mitochondria (MitoTracker DeepRed). Control drosophila (w-1118) show only mitochondria staining but no GFP signal. Hoechst stains nuclei. In FIG. 19, the study confirms by PCR and sequencing, the full length mtON::GFP product is present and has the correct nucleotide sequence.

The results disclosed herein that increases in lifespan are achieved by energizing mitochondria are an unexpected result.

While various embodiments have been described above, it should be understood that such disclosures have been presented by way of example only and are not limiting. Thus, the breadth and scope of the subject compositions and methods should not be limited by any of the above-described exemplary embodiments, but should be defined only in accordance with the following claims and their equivalents.

The above description is for the purpose of teaching the person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present invention, which is defined by the following claims. The claims are intended to cover the components and steps in any sequence which is effective to meet the objectives there intended, unless the context specifically indicates the contrary.

1-20. (canceled)

21. A method of ameliorating age-related mitochondrial dysfunction in a subject, comprising the steps of:
expressing a fusion protein in target cells in the subject, wherein the fusion protein comprises a first moiety that targets the fusion protein to the mitochondrial inner membrane, and a second moiety that comprises a light-activated proton pump, wherein the first moiety orients the light-activated proton pump in the direction to pump protons from the mitochondrial matrix to the inner membrane space;
exposing the target cells to light to activate the proton pump to increase the protonmotive force (PMF) across the mitochondrial inner membrane,
wherein increased PMF in the mitochondria of the target cells reverses mitochondrial dysfunction in the target cells.

22. A method of ameliorating mitochondrial dysfunction in a subject, comprising the steps of:
expressing a fusion protein in target cells in the subject, wherein the fusion protein comprises a first moiety that targets the fusion protein to the mitochondrial inner membrane, and a second moiety that comprises a light-activated proton pump, wherein the first moiety orients the light-activated proton pump in the direction to pump protons from the mitochondrial matrix to the inner membrane space;
exposing the target cells to light to activate the proton pump to increase the protonmotive force (PMF) across the mitochondrial inner membrane,
wherein increased PMF in the mitochondria of the target cells reverses mitochondrial dysfunction in the target cells, wherein the target cells comprise hair follicle cells or keratinocytes.

23. A method of treating cancer in a subject, comprising the steps of:
expressing a fusion protein in T cells in the subject, wherein the fusion protein comprises a first moiety that targets the fusion protein to the mitochondrial inner membrane, and a second moiety that comprises a light-activated proton pump, wherein the first moiety orients the light-activated proton pump in the direction to pump protons from the mitochondrial matrix to the inner membrane space;
exposing fusion protein-expressing T cells to light to activate the proton pump to increase the protonmotive force (PMF) across the mitochondrial inner membrane,
wherein increased PMF in the mitochondria of the fusion protein-expressing T cells enhances cancer killing activity of the T cells.

24. The method of claim 23, wherein the T cells are chimeric antigen receptor (CAR)-T cells.

25. The method of claim 23, wherein the T cells are CD8+ T cells.

26. The method of claim 21, wherein the first moiety comprises SEQ ID NO:6.

27. The method of claim 21, wherein the second moiety comprises SEQ ID NO: 10.

28. The method of claim 21, wherein the first moiety comprises an amino acid sequence that is at least 95% homologous to SEQ ID NO:6 and wherein the second moiety comprises an amino acid sequence that is at least 80% homologous to SEQ ID NO: 10.

29. The method of claim 21, wherein the fusion protein comprises the amino acid sequence of SEQ ID NO: 11.

30. The method of claim 22, wherein the first moiety comprises SEQ ID NO:6.

31. The method of claim 22, wherein the second moiety comprises SEQ ID NO: 10.

32. The method of claim 22, wherein the first moiety comprises an amino acid sequence that is at least 95% homologous to SEQ ID NO:6 and wherein the second moiety comprises an amino acid sequence that is at least 80% homologous to SEQ ID NO: 10.

33. The method of claim 22, wherein the fusion protein comprises the amino acid sequence of SEQ ID NO: 11.


SEQUENCE LISTING (27 sequences)

SEQ ID NO: 1 - protein, 758 aa, Homo sapiens
  SITE (1)..(758): Human IMMT (UniProtKB - Q16891)
MLRACQLSGVTAAAQSCLCGKFVLRPLRPCRRYSTSGSSGLTTGKIAGAGLLFVGGGIGGTILYAKWDSHFRESVEKTIPYSDKLFEMVLGPAAYNVPLPKKSIQSGPLKISSVSEVMKESKQPASQLQKQKGDTPASATAPTEAAQIISAAGDTLSVPAPAVQPEESLKTDHPEIGEGKPTPALSEEASSSSIRERPPEEVAARLAQQEKQEQVKIESLAKSLEDALRQTASVTLQAIAAQNAAVQAVNAHSNILKAAMDNSEIAGEKKSAQWRTVEGALKERRKAVDEAADALLKAKEELEKMKSVIENAKKKEVAGAKPHITAAEGKLHNMIVDLDNVVKKVQAAQSEAKVVSQYHELVVQARDDFKRELDSITPEVLPGWKGMSVSDLADKLSTDDLNSLIAHAHRRIDQLNRELAEQKATEKQHITLALEKQKLEEKRAFDSAVAKALEHHRSEIQAEQDRKIEEVRDAMENEMRTQLRRQAAAHTDHLRDVLRVQEQELKSEFEQNLSEKLSEQELQFRRLSQEQVDNFTLDINTAYARLRGIEQAVQSHAVAEEEARKAHQLWLSVEALKYSMKTSSAETPTIPLGSAVEAIKANCSDNEFTQALTAAIPPESLTRGVYSEETLRARFYAVQKLARRVAMIDETRNSLYQYFLSYLQSLLLFPPQQLKPPPELCPEDINTFKLLSYASYCIEHGDLELAAKFVNQLKGESRRVAQDWLKEARMTLETKQIVEILTAYASAVGIGTTQVQPE

SEQ ID NO: 2 - protein, 65 aa, Homo sapiens
  SITE (1)..(65): first 65 aa of human IMMT sequence
MLRACQLSGVTAAAQSCLCGKFVLRPLRPCRRYSTSGSSGLTTGKIAGAGLLFVGGGIGGTILYA

SEQ ID NO: 3 - protein, 187 aa, Homo sapiens
  SITE (1)..(187): first 187 aa of human IMMT sequence
MLRACQLSGVTAAAQSCLCGKFVLRPLRPCRRYSTSGSSGLTTGKIAGAGLLFVGGGIGGTILYAKWDSHFRESVEKTIPYSDKLFEMVLGPAAYNVPLPKKSIQSGPLKISSVSEVMKESKQPASQLQKQKGDTPASATAPTEAAQIISAAGDTLSVPAPAVQPEESLKTDHPEIGEGKPTPALSE

SEQ ID NO: 4 - protein, 757 aa, Mus musculus
  SITE (1)..(757): Mouse IMMT (UniProtKB - Q8CAQ8)
MLRACQLSGVTVAAQSCLCGKFVLRPLRPCRRYSTSSSSGLTAGKIAGAGLLFVGGGIGGTILYAKWDSHFRESVEKTIPYSDKLFGMVLGSAPYTVPLPKKPVQSGPLKISSVSEVMKDSKLPVAQSQKTKGDTPASAASTGAAQIISAAGDTLSVPAPAVQHEDTIKTECPNTNEGKSTSETTEEAFSSSVRERPPEEVAARLAQQEKQEQVEMESLAKSLEDALNRTSSVTLQTITAQNAAVQAVKAHSNILKTAMDNSEIAGEKKSAQWRTVEGALKERRKAVDEAADALLKAKEELEKMKTIIEDAKKREIAGATPHITAAEGRLHNMIVDLDNVVKKVQAAQSEAKVVSQYHELVVQARDDFRKELDSITPDITPGWKGMSISDLAGKLSTDDLNSLIAHAHRRIDQLNRELAQQKATEKQHIELALEKHKLEEKRTFDSAVAKALEHHRSEIQAEQDRKVEEVRDAMENEMRTQLRRQAAAHTDHLRDVLKVQEQELKYEFEQGLSEKLSEQELEFRRRSQEQMDSFTLDINTAYARLRGIEQAVQSHAVAEEEARKAHQLWLSVEALKYSMKTSSAEMPTIPLGSAVEAIRVNCSDNEFTQALTAAIPPESLTRGVYSEETLRARFYAVQKLARRVAMIDETRNSLYQYFLSYLQSLLLFPPKQLKPPAELYPEDINTFKLLSYASYCIEHGDLELAAKFVNQLKGESRRVAQDWLKEARMTLETKQIVEILTAYASAVGIGTTQVQQE

SEQ ID NO: 5 - protein, 65 aa, Mus musculus
  SITE (1)..(65): first 65 aa of mouse IMMT sequence
MLRACQLSGVTVAAQSCLCGKFVLRPLRPCRRYSTSSSSGLTAGKIAGAGLLFVGGGIGGTILYA

SEQ ID NO: 6 - protein, 187 aa, Mus musculus
  SITE (1)..(187): first 187 aa of mouse IMMT sequence
MLRACQLSGVTVAAQSCLCGKFVLRPLRPCRRYSTSSSSGLTAGKIAGAGLLFVGGGIGGTILYAKWDSHFRESVEKTIPYSDKLFGMVLGSAPYTVPLPKKPVQSGPLKISSVSEVMKDSKLPVAQSQKTKGDTPASAASTGAAQIISAAGDTLSVPAPAVQHEDTIKTECPNTNEGKSTSETTEE

SEQ ID NO: 7 - protein, 609 aa, Rattus norvegicus
  SITE (1)..(609): Rat IMMT (UniProtKB - Q3KR86); would use aa 1-187; minimum aa 1-66)
MLRACQLSGVTVAAQSCLCGKFVLRPLRPCRRYSTSSSSGVTAGKIAGAGLLFVGGGIGGTILYAKWDSHFRESVEKTIPYSDKLFGMVLGSAPYTVPLPKKPIQSGPLKISSVSEVMTDSELPMAQTQETNGDTPASAAGDPAPEVEHEDTINTECPNTDEGTSTFVTAALAKSLEDALNQTATVTRQTITAQNAAVQAVKAHSSTLKTAMDNSEIAGEKKSAQWRTVEGALKERRKAVDEAADALLKAKEELEKMKTIIEDAKKREIAGATPYITAAEEKLHSMIVDLDSVVKKVQAAQSEAKVVSQYHELVVQARDDFRKELDSITPDITPGWKGMSISDLAGTLSTDDLNALIAHAHRRIDQLNRELAQQKATEKQHIELALERQKLEEKRAFDSAVAKALEHHRSEIQAEQDRKVEEVRDAMENEMRTQLRRQAAAHTDHLRDVLKVQEQELKFEFEQDLSEKLSEQELEFHRRSQEQMDNFTLDINTAYARLRGIEQAVQSHAVAEEEARKAHQLWLSVEALKYSMKTSSAEMPTIPLGSAVEAIRVSCSDNEFTQALTAAIPPESLTRGVYSEETLRARFYAVQKLAGRVAMIDETKNSLYQ

SEQ ID NO: 8 - protein, 66 aa, Rattus norvegicus
  SITE (1)..(66): first 66 aa of rat IMMT sequence
MLRACQLSGVTVAAQSCLCGKFVLRPLRPCRRYSTSSSSGVTAGKIAGAGLLFVGGGIGGTILYAK

SEQ ID NO: 9 - protein, 187 aa, Rattus norvegicus
  SITE (1)..(187): first 187 aa of rat IMMT sequence
MLRACQLSGVTVAAQSCLCGKFVLRPLRPCRRYSTSSSSGVTAGKIAGAGLLFVGGGIGGTILYAKWDSHFRESVEKTIPYSDKLFGMVLGSAPYTVPLPKKPIQSGPLKISSVSEVMTDSELPMAQTQETNGDTPASAAGDPAPEVEHEDTINTECPNTDEGTSTFVTAALAKSLEDALNQTATVT

SEQ ID NO: 10 - protein, 314 aa, L. maculans
  SITE (1)..(314): Mac (L. maculans) sequence
LIVDQFEEVLMKTSQLFPLPTATQSAQPTHVAPVPTVLPDTPIYETVGDSGSKTLWVVFVLMLIASAAFTALSWKIPVNRRLYHVITTIITLTAALSYFAMATGHGVALNKIVIRTQHDHVPDTYETVYRQVYYARYIDWAITTPLLLLDLGLLAGMSGAHIFMAIVADLIMVLTGLFAAFGSEGTPQKWGWYTIACIAYIFVVWHLVLNGGANARVKGEKLRSFFVAIGAYTLILWTAYPIVWGLADGARKIGVDGEIIAYAVLDVLAKGVFGAWLLVTHANLRESDVELNGFWANGLNREGAIRIGEDDGAR

SEQ ID NO: 11 - protein, 504 aa, Artificial Sequence
  Synthetic Complete mtON fusion protein sequence
MLRACQLSGVTVAAQSCLCGKFVLRPLRPCRRYSTSSSSGLTAGKIAGAGLLFVGGGIGGTILYAKWDSHFRESVEKTIPYSDKLFGMVLGSAPYTVPLPKKPVQSGPLKISSVSEVMKDSKLPVAQSQKTKGDTPASAASTGAAQIISAAGDTLSVPAPAVQHEDTIKTECPNTNEGKSTSETTEEPAGLIVDQFEEVLMKTSQLFPLPTATQSAQPTHVAPVPTVLPDTPIYETVGDSGSKTLWVVFVLMLIASAAFTALSWKIPVNRRLYHVITTIITLTAALSYFAMATGHGVALNKIVIRTQHDHVPDTYETVYRQVYYARYIDWAITTPLLLLDLGLLAGMSGAHIFMAIVADLIMVLTGLFAAFGSEGTPQKWGWYTIACIAYIFVVWHLVLNGGANARVKGEKLRSFFVAIGAYTLILWTAYPIVWGLADGARKIGVDGEIIAYAVLDVLAKGVFGAWLLVTHANLRESDVELNGFWANGLNREGAIRIGEDDGAR

SEQ ID NO: 12 - DNA, 1512 bp, Artificial Sequence
  Synthetic mtON nucleotide sequence
atgctgcgggcctgtcagttatccggcgtgaccgtcgcggcccagagttgtctgtgtgggaagttcgtcctccgtccactacgaccatgccgtagatactccacttcaagcagttctgggttgactgctggtaagattgctggagctggccttttgttcgttggtggagggattggtggcactatcctgtatgccaaatgggattcccatttccgggaaagtgtagagaaaaccatcccttactcagacaaactctttgggatggttcttggttctgcgccttatactgttccattaccaaagaaaccggttcagtctggtccactaaaaatctccagtgtatcagaggtgatgaaagactccaaactgcctgtggcacagagccagaaaacgaagggagacactccagcttcagcagcatctacaggggcagctcaaattatttctgcagcaggtgatacactttcggtcccggcccctgcagttcagcatgaggacactataaaaaccgaatgtcccaacactaatgaaggaaaatctacgtctgaaaccacagaggaacctgcaggcttgatcgtggaccagttcgaggaggtgctgatgaagaccagccagctgttcccactgccaaccgctacccagagcgcccagccaacccacgtggcccccgtgccaaccgtgctgcccgacacccccatctacgagaccgtgggcgacagcggcagcaagaccctgtgggtggtgttcgtgctgatgctgatcgccagcgccgccttcaccgccctgagctggaagatccccgtgaacaggaggctgtaccacgtgatcaccaccatcatcaccctgaccgccgccctgagctacttcgctatggctaccggccacggagtggccctgaacaagatcgtgatcaggacccagcacgaccacgtgcccgacacctacgagaccgtgtaccgacaggtgtactacgccaggtacatcgactgggctatcaccaccccactgctgctgctggacctgggactgctggctggaatgagcggagcccacatcttcatggccatcgtggctgacctgatcatggtgctgaccggcctgttcgctgctttcggcagcgagggaaccccacagaagtggggatggtacaccatcgcctgcatcgcctacatcttcgtggtgtggcacctggtgctgaacggcggcgccaacgccagggtgaagggcgagaagctgaggagcttcttcgtggccatcggagcttacaccctgatcctgtggaccgcttacccaatcgtgtggggactggctgacggagctaggaagatcggagtggacggagagatcatcgcttacgctgtgctggacgtgctggctaagggagtgttcggagcttggctgctggtgacccacgccaacctgagggagagcgacgtggagctgaacggcttctgggccaacggcctgaacagggagggcgccatcaggatcggcgaggacgacggcgcccga

SEQ ID NO: 13 - DNA, 32 bp, Artificial Sequence
  Synthetic Forward amplification primer for Mac-GFP
acacctgcaggcttgatcgtggaccagttcga

SEQ ID NO: 14 - DNA, 32 bp, Artificial Sequence
  Synthetic Reverse amplification primer for Mac-GFP
cacagcggccgcttacttgtacagctcgtcca

SEQ ID NO: 15 - DNA, 33 bp, Artificial Sequence
  Synthetic Forward amplification primer for Immt1
acaaccggtaaaaatgctgcgggcctgtcagtt

SEQ ID NO: 16 - DNA, 31 bp, Artificial Sequence
  Synthetic Reverse amplification primer for Immt1
caccctgcaggttcctctgtggtttcagacg

SEQ ID NO: 17 - DNA, 27 bp, Artificial Sequence
  Synthetic Forward amplification primer for promoter Peft-3 (also known as Peef-1A.1)
aacaaagcttgcacctttggtctttta

SEQ ID NO: 18 - DNA, 26 bp, Artificial Sequence
  Synthetic Reverse amplification primer for promoter Peft-3 (also known as Peef-1A.1)
acatctagagagcaaagtgtttccca

SEQ ID NO: 19 - DNA, 23 bp, Artificial Sequence
  Synthetic Forward amplification primer for promoter Pmyo-3
acagctagctgtgtgtgattgct

SEQ ID NO: 20 - DNA, 26 bp, Artificial Sequence
  Synthetic Reverse amplification primer for promoter Pmyo-3
acaaccggtgcggcaattctagatgg

SEQ ID NO: 21 - DNA, 101 bp, Artificial Sequence
  Synthetic Forward amplification primer for red fluorescent protein mCherry
ctcgcccagcttcttgtggctcacgccgccgtctcatcgattcgctcaacttatcctcgtagccctcggactcctcgtagcatggtctcaaagggtgaaga

SEQ ID NO: 22 - DNA, 59 bp, Artificial Sequence
  Synthetic Reverse amplification primer for red fluorescent protein mCherry
ttttaaaaaacggtgacgcaagacaatcaattgttctacttatacaattcatccatgcc

SEQ ID NO: 23 - DNA, 20 bp, Artificial Sequence
  Synthetic DNA target of crRNA
ctaataagttgagcgaatcg

SEQ ID NO: 24 - DNA, 20 bp, Artificial Sequence
  Synthetic Forward amplification primer for uaDf5 mitochondrial DNA
ttggtgttacaggggcaaca

SEQ ID NO: 25 - DNA, 24 bp, Artificial Sequence
  Synthetic Reverse amplification primer for uaDf5 mitochondrial DNA
cttctacagtgcattgacctagtc

SEQ ID NO: 26 - DNA, 20 bp, Artificial Sequence
  Synthetic Forward amplification primer for uaDf5 DNA
ccatccgtgctagaagacaa

SEQ ID NO: 27 - DNA, 24 bp, Artificial Sequence
  Synthetic Reverse amplification primer for uaDf5 DNA
cttctacagtgcattgacctagtc